(19) United States
(12) Patent Application Publication
(10) Pub. No.: US 2020/0392562 A1
(43) Pub. Date: Dec. 17, 2020

(54) POLYMERASE CHAIN REACTION COMPOSITION COMPRISING AMINES

(71) Applicant: THERMO FISHER SCIENTIFIC BALTICS UAB, Carlsbad, CA (US)

(72) Inventors: Karolis MATJOSAITIS, Vilnius (LT); Renaldas RIMKUS, Vilnius (LT); Laurynas VANAGAS, Vilnius (LT); Rasa SUKACKAITE, Vilnius (LT); Skaiste VALINSKYTE, Kazlu Rüda (LT); Edita ELIJOSIUTE, Vilnius (LT); Dovile STREPETKAITE, Vilnius (LT)

(21) Appl. No.: 16/954,979

(22) PCT Filed: Dec. 20, 2018
(86) PCT No.: PCT/EP2018/086321

§ 371 Date: Jun. 27, 2019

Related U.S. Application Data
(60) Provisional application No. 62/610,042, filed on Dec. 22, 2017.

(51) Int. Cl. C12Q 1/686; C12Q 1/6806

(52) U.S. Cl. CPC C12Q 1/686; C12Q 1/6806; C12Q 2531/113; C12Q 2527/125; C12Q 2521/119; C12Q 2521/101; C12Q 2521/107

(57) ABSTRACT

Compositions, methods, and kits comprising amines are described for use in nucleic acid synthesis. In some embodiments, amines improve nucleic acid synthesis product yield or tolerance to inhibitors of nucleic acid synthesis.

### CROSS-REFERENCE

This application is a 371 U.S. National Phase Application of PCT/EP2018/086321 filed Dec. 20, 2018 (now published as WO2019/0122200), which application claims the benefit under 35 U.S.C. § 119(e) of U.S. Provisional Application No. 62/610,042 filed Dec. 22, 2017. The entire contents of the aforementioned applications are incorporated by reference herein.

### SEQUENCE LISTING

The instant application contains a Sequence Listing which has been submitted electronically in ASCII format and is hereby incorporated by reference in its entirety. Said ASCII copy, created on Nov. 19, 2018, is named LT01310PCT_SL.txt and is 1,267 bytes in size.

### FIELD

This application relates to the field of compositions and methods for synthesis of nucleic acids.

### BACKGROUND

Thermophilic DNA polymerases are commonly used in biotechnology and molecular biology applications, including nucleic acid synthesis techniques such as amplification (e.g., PCR), which involves cycles of alternating denaturation and primer annealing and extension. Thermophilic DNA polymerases are resistant to inactivation by high temperatures and so are compatible with thermal denaturation steps. Amplification yield depends on thermophilic DNA polymerase performance parameters, such as synthesis speed, processivity, and thermostability.

Nucleic acid synthesis inhibitors may decrease PCR yield or completely abolish amplification if present at higher concentrations. PCR inhibitors may be present in the original sample, such as blood, fabrics, tissues and soil, and/or may be added as a result of the sample processing and nucleic acid extraction steps (see Schrader et al., Journal of Applied Microbiology 113:1014-1026 (2012) and Alaeddini Forensic Science International: Genetics 6:297-305 (2012)). Examples of common nucleic acid synthesis inhibitors include polyanions such as heparin or xylan; chaotropic agents as sodium dodecyl sulfate or urea; certain organic compounds such as humic acid or bile salts; different proteins such as collagen, heme and heme-containing proteins; metal ions such as calcium; chelators such as citrate or EDTA; organic solvents such as ethanol or isopropanol; nucleic acid intercalating dyes; and the like. Nucleic acid synthesis reaction may be inhibited in the presence of magnetic beads. Improved tolerance for PCR inhibitors reduces the need for additional purification and other sample processing steps, reduces the frequency of unsatisfactory synthesis reactions, and broadens the spectrum of samples that can be successfully amplified.

Certain thermophilic DNA polymerases can show higher tolerance for inhibitors than other wild type enzymes. Tolerance for PCR inhibitors may be further increased by protein engineering. Thermophilic DNA polymerase variants with increased inhibitor tolerance were obtained both by point mutagenesis (US20130034879A1, US20170204384A1) and N-terminal deletions (Kermekchiev et al., Nucleic Acids Res 37(5):e40 (2009)). Certain artificial DNA polymerases comprise a fused non-specific double-stranded DNA (dsDNA) binding domain. The presence of this domain improves performance of the enzyme with respect to inhibitor tolerance (US20040002076A1). Another strategy to improve inhibitor tolerance is based on protein additives. Bovine serum albumin (BSA), gp32 or gelatin are known to act as scavengers, which can relieve inhibition in PCR (Kreader Appl Environ Microbiol 62(3): 1102-1106 (1996) and US20120244527A1). Even when polymerases with increased inhibitor tolerance are used, further inhibitor tolerance may be desired.

Provided herein are compounds that increase tolerance for nucleic acid synthesis reaction inhibitors when used in a reaction composition with DNA or RNA polymerases.

### SUMMARY

Described herein are compositions and kits comprising amines for use in synthesizing nucleic acids. Further, methods of use are described wherein amines improve nucleic acid synthesis product yield and/or tolerance to inhibitors of nucleic acid synthesis. These amines may improve tolerance to inhibitors that are inherently present in the sample or that were added in upstream processing steps.

In accordance with the description, methods of improving nucleic acid synthesis product yield and/or tolerance to inhibitors during nucleic acid synthesis from a nucleic acid template comprising mixing a sample comprising the nucleic acid template with a composition comprising one or more amines of formula I:

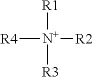

or salts thereof wherein R1 is H; R2 is chosen from alkyl, alkenyl, alkynyl, or (CH2)n-R5, wherein n=1 to 3, and R5 is aryl, amino, thiol, mercaptan, phosphate, hydroxy, or alkoxy; and R3 and R4 may be the same or different and are independently chosen from H or alkyl, with the proviso that if R2 is (CH2)n-R5, then at least one of R3 and/or R4 is alkyl; providing an enzyme for synthesizing nucleic acid molecules; and incubating said mixture under conditions suitable for synthesis of a nucleic acid molecule complementary to all or a portion of said template.

Also embodied is a kit for use in synthesis of a nucleic acid molecule, wherein the kit provides increased yield or tolerance to inhibitors, said kit comprising, (i) one or more enzymes for synthesizing nucleic acid molecules or instructions to provide one or more enzymes for synthesizing nucleic acid molecules and (ii) one or more amines of formula I:

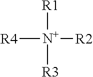

or salts thereof wherein R1 is H; R2 is chosen from alkyl, alkenyl, alkynyl, or (CH2)n-R5, wherein n=1 to 3, and R5 is aryl, amino, thiol, mercaptan, phosphate, hydroxy, or alkoxy; and R3 and R4 may be the same or different and are independently chosen from H or alkyl, with the proviso that if R2 is (CH2)n-R5, then at least one of R3 and/or R4 is alkyl.

Also embodied is a composition for improving nucleic acid synthesis product yield or tolerance to inhibitors of nucleic acid synthesis comprising one or more enzymes for synthesizing nucleic acid molecules and one or more amines of formula I:

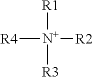

or salts thereof wherein R1 is H; R2 is chosen from alkyl, alkenyl, alkynyl, or (CH2)n-R5, wherein n=1 to 3, and R5 is aryl, amino, thiol, mercaptan, phosphate, hydroxy, or alkoxy; and R3 and R4 may be the same or different and are independently chosen from H or alkyl, with the proviso that if R2 is (CH2)n-R5, then at least one of R3 and/or R4 is alkyl.

In some embodiments, the one or more amines of formula I and the one or more enzymes for synthesizing nucleic acid molecules are provided in a single composition.

In some embodiments, the the one or more amines of formula I and the one or more enzymes for synthesizing nucleic acid molecules are provided in separate compositions.

In some embodiments, the one or more amines of formula I and the one or more enzymes for synthesizing nucleic acid molecules are provided simultaneously.

In some embodiments, the synthesis is for amplification.

In some embodiments, the one or more amines of Formula I and/or the one or more enzymes for synthesizing nucleic acid molecules is in a stabilized formulation for long-term storage.

In some embodiments, the one or more amines of Formula I and/or the one or more enzymes for synthesizing nucleic acid molecules are provided in a formulation that comprises a stabilizer and/or detergent.

In some embodiments, the sample comprises one or more nucleic acid synthesis inhibitors.

In some embodiments, the nucleic acid synthesis inhibitor is a polyanion. In some embodiments, the polyanion is heparin or xylan.

In some embodiments, the nucleic acid synthesis inhibitor is a chaotropic agent. In some embodiments, the chaotropic agent is sodium dodecyl sulfate or urea.

In some embodiments, the nucleic acid synthesis inhibitor is a protein. In some embodiments, the inhibitor is collagen, heme or heme-containing proteins.

In some embodiments, the nucleic acid synthesis inhibitor is an organic compound. In some embodiments, the inhibitor is humic acid, or bile salts.

In some embodiments, the nucleic acid synthesis inhibitor is a chelator. In some embodiments, the chelator is citrate or EDTA.

In some embodiments, the nucleic acid synthesis inhibitor is an organic solvent. In some embodiments, the solvent is ethanol or isopropanol.

In some embodiments, the nucleic acid synthesis inhibitor is a nucleic acid intercalating dye.

In some embodiments, the nucleic acid synthesis is performed in the presence of microcarriers. Microcarriers may be magnetic, i.e. comprise material that responds to a magnetic field, such as, but not limited to ferromagnetic, paramagnetic, and supermagnetic materials. Exemplary magnetic microcarriers are magnetic beads. In some embodiments, the beads are carboxylated magnetic beads. In some embodiments, the carboxylated magnetic beads are Agencourt® AMPure® XP (Beckman Coulter, Inc), Sera-Mag™ SpeedBeads™ (GE Healthcare Life Sciences), MyOne carboxylated beads (DynaBeads), or Mag-Bind® RXNPure (Omega Bio-tek, Inc) beads. In some embodiments, magnetic beads present in the sample were used in a prior purification step.

In some embodiments, the nucleic acid synthesis inhibitor is a metal ion. In some embodiments, the metal ion is calcium.

In some embodiments, the composition or kit further comprises one or more additional components chosen from (i) one or more nucleic acid molecules; (ii) one or more nucleotides; (iii) one or more buffering salts; and (iv) one or more cofactors.

In some embodiments, the one or more nucleic acid molecules comprise RNA or DNA. In some embodiments, the RNA or DNA comprise a primer for a synthesis reaction.

In some embodiments, the one or more nucleotides comprise dNTPs or NTPs. In some embodiments, the one or more buffering salts comprise acetate, sulfate, hydrochloride, or phosphate or free acid forms of Tris-(hydroxymethyl)aminomethane (TRIS®). In some embodiments, the one or more cofactor comprises a magnesium salt.

In some embodiments, the composition or kit further comprises one or more additional additives. In some embodiments, the additional additive comprises a salt. In some embodiments, the additional salt comprises a potassium salt. In some embodiments, the potassium salt comprises potassium chloride (KCl). In some embodiments, the KCl concentration of the composition may be reduced or KCl may be omitted based on the presence of an amine.

In some embodiments, the additional additive comprises a detergent. In some embodiments, the detergent comprises Hecameg (6-0-(N-Heptylcarbamoyl)-methyl-a-D-glucopyranoside), Triton X-200, Brij-58, CHAPS, n-Dodecyl-b-D-maltoside, NP-40, sodium dodecyl sulfate (SDS), TRITON® X-15, TRITON® X-35, TRITON® X-45, TRITON® X-100, TRITON® X-102, TRITON® X-114, TRITON® X-165, TRITON® X-305, TRITON® X-405, TRITON® X-705, Tween® 20 and/or ZWITTERGENT®.

In some embodiments, the additional additive comprises at least one protein stabilizer. In some embodiments, the protein stabilizer comprises bovine serum albumin (BSA), an inactive polymerase, or apotransferrin.

In some embodiments, the additional additive comprises at least one reducing agent. In some embodiments, the reducing agent comprises dithiothreitol (DTT).

In some embodiments, the additional additive comprises an agent that enhances nucleic acid synthesis from high GC-content templates. In some embodiments, the high GC-content is about 65% or more. In some embodiments, the agent that enhances nucleic acid synthesis from high GC-content templates comprises ethylene glycol, polyethylene glycol, 1,2-propanediol, ammonium sulfate, dimethyl sulfoxide (DMSO), glycerol, formamide, 7-deaza-GTP, acetamide, or betaine.

In some embodiments, the additional additive comprises a dye. In some embodiments, the dye comprises xylene cyanol FF, tartrazine, phenol red, quinoline yellow, Brilliant Blue, Patent Blue, indigocarmine, acid red 1, m-cresol purple, amaranth, cresol red, neutral red, bromocresol green, acid violet 5, bromo phenol blue, or orange G.

In some embodiments, the additional additive comprises glycerol, trehalose, lactose, maltose, galactose, glucose, sucrose, dimethyl sulfoxide (DMSO), polyethylene glycol, or sorbitol.

In some embodiments, the composition comprises a hot start composition.

In some embodiments, the one or more enzyme for synthesizing nucleic acid is chosen from a DNA polymerase, an RNA polymerase, or a reverse transcriptase. In some embodiments, the DNA polymerase comprises Phi29 or its derivatives, Bsm, Bst, T4, T7, DNA Pol I, or Klenow Fragment; or mutants, variants and derivatives thereof.

In certain embodiments, a polymerase comprises a fragment or variant of an A, B, C, D, X, or Y polymerase having polymerase activity. In certain embodiments, a polymerase comprises a family A DNA polymerase, or a fragment or variant thereof having polymerase activity. In certain such embodiments, the family A polymerase is a bacterial family A polymerase, such as a polymerase from the genus Bacillus, Thermus, Rhodothermus or Thermotoga. The family A polymerase may be thermophilic. In certain such embodiments, the family A polymerase is Taq DNA polymerase (UniProtKB: P19821) or a fragment or variant thereof having polymerase activity. In certain embodiments, a variant of Taq DNA polymerase comprises an amino acid sequence having at least 80%, 85%, 90%, 95%, 96%, 97%, 98%, or 99% identity to Taq DNA polymerase. Exemplary variants of Taq DNA polymerase are described in e.g. U.S. Pat. No. 9,493,848B2, U.S. Pat. Nos. 6,395,524, 6,602,695, 5,614,365, 5,466,591, Brandis et al., 1998, Barnes and Kermekchiev, 2000, Kermekchiev and Barnes, 2004; Kermekchiev and Kirilova, 2006, Kermekchiev et al, 2009; Zhang et al, 2010.

In certain embodiments, a polymerase comprises a family B DNA polymerase or a fragment or variant thereof having polymerase activity. In certain such embodiments, the family B polymerase is an archaeal family B polymerase, such as a polymerase from the genus Thermococcus, Pyrococous, or Pyrobaculum. Such polymerases are thermophilic. In certain such embodiments, the family B polymerase is Pfu DNA polymerase (UniProtKB: P61875) or a fragment or variant thereof having polymerase activity. In certain embodiments, a variant of Pfu DNA polymerase or a Pfu-like polymerase comprises an amino acid sequence having at least 80%, 85%, 90%, 95%, 96%, 97%, 98%, or 99% identity to Pfu DNA polymerase.

In some embodiments, the DNA polymerase comprises a thermophilic DNA polymerase. In some embodiments, the thermophilic DNA polymerase comprises Taq, Tbr, Tfl, Tth, Tli, Tfi, Tne, Tma, Pfu, Pwo, Kod, VENT™, DEEPVENT™ DNA polymerase; Phusion DNA polymerase (U.S. Pat. No. 7,560,260B2, U.S. Pat. No. 8,415,129B2, U.S. Pat. No. 6,228,628B1); Phusion U DNA polymerase; SuperFi DNA polymerase; SuperFi U DNA Polymerase (as described in 62/524,730; US20170204384A1) or mutants, variants and derivatives thereof; and/or GoTaq G2 Hot Start Polymerase (Promega), OneTaq® Hot Start DNA Polymerase (NEB), TaKaRa Taq™ DNA Polymerase Hot Start (TaKaRa), KAPA2G Robust HotStart DNA Polymerase (KAPA), FastStart Taq DNA Polymerase (Roche), HotStart Taq DNA Polymerase (Qiagen), Q5 DNA Polymerase, Kapa HiFi DNA Polymerase, PrimeStar Max DNA Polymerase, PrimeStar GXL DNA Polymerase.

In some embodiments, the DNA polymerase comprises a chimeric DNA polymerase. In some embodiments, the chimeric DNA polymerase comprises a sequence nonspecific double stranded DNA (dsDNA) binding domain. For example, the the chimeric DNA polymerase comprises a Pfu-like polymerase fused to a sequence nonspecific double stranded DNA (dsDNA) binding domain, where the Pfu-like polymerase has at least 80%, 85%, 90%, 95%, 96%, 97%, 98%, or 99% identity to Pfu DNA polymerase. Exemplary polymerases are Phusion DNA polymerase; Phusion U DNA polymerase; SuperFi DNA polymerase; SuperFi U DNA Polymerase, Q5 DNA Polymerase. In some embodiments, the dsDNA binding domain comprises Sso7d from Sulfolobus solfataricus; Sac7d, Sac7a, Sac7b, and Sac7e from S. acidocaldarius; and Ssh7a and Ssh7b from Sulfolobus shibatae; Pae3192; Pae0384; Ape3192; HMf family archaeal histone domains; or an archaeal proliferating-cell nuclear antigen (PCNA) homolog.

In some embodiments, the RNA polymerase comprises SP6, T7, or T3 RNA polymerase, or mutants, variants, or derivatives thereof.

In some embodiments, the reverse transcriptase comprises M-MLV reverse transcriptase, RSV reverse transcriptase, AMV reverse transcriptase, RAV reverse transcriptase, MAV reverse transcriptase, HIV reverse transcriptase, and/or mutants, variants, and derivatives thereof; and/or SuperScript II reverse transcriptase, SuperScript III reverse transcriptase, SuperScript IV reverse transcriptase, Maxima reverse transcriptase, GoScript reverse transcriptase, PrimeScript reverse transcriptase, iScript reverse transcriptase, Sensiscript reverse transcriptase, ProtoScript reverse transcriptase, AffinityScript Reverse Transcriptase, NxtScript Reverse Transcriptase, RnaUsScript Reverse Transcriptase, RocketScript Reverse Transcriptase, GoScript Reverse Transcriptase, and/or Thermoscript reverse transcriptase

In some embodiments, the method is for polymerase chain reaction (PCR).

In some embodiments, R2 is an alkyl. In some embodiments, the alkyl is a C1-C5 (branched or linear) alkyl. In some embodiments, the alkyl is a C1-C3 alkyl. In some embodiments, the alkyl is methyl.

In some embodiments, R3 and/or R4 is an H.

In some embodiments, R3 and/or R4 is an alkyl. In some embodiments, the alkyl is a C1-C5 (branched or linear) alkyl. In some embodiments, the alkyl is a C1-C3 alkyl. In some embodiments, the alkyl is methyl.

In some embodiments, said composition or kit comprises a salt form of the one or more amines of formula I. In some embodiments, the salt form comprises a chloride, sulfate, or acetate salt.

In some embodiments, said composition or kit comprises one amine of formula I or salts thereof.

In some embodiments, said composition or kit comprises at least two amines of formula I or salts thereof.

In some embodiments, said composition or kit comprises at least three amines of formula I or salts thereof.

In some embodiments, said composition or kit comprises at least four amines of formula I or salts thereof.

In some embodiments, the concentration of the one or more amines is 10-250 mM. In some embodiments, the concentration of the one or more amines is 50-110 mM.

In some embodiments, at least one amine of formula I comprises dimethylamine hydrochloride. In some embodiments, the concentration of dimethylamine hydrochloride is 10-250 mM. In some embodiments, the concentration of dimethylamine hydrochloride is 50-110 mM.

In some embodiments, at least one amine of formula I comprises diethylamine hydrochloride. In some embodiments, the concentration of diethylamine hydrochloride is 10-250 mM. In some embodiments, the concentration of diethylamine hydrochloride is 50-110 mM.

In some embodiments, at least one amine of formula I comprises diisopropylamine hydrochloride. In some embodiments, the concentration of diisopropylamine hydrochloride is 10-250 mM. In some embodiments, the concentration of diisopropylamine hydrochloride is 50-110 mM.

In some embodiments, at least one amine of formula I comprises ethyl(methyl)amine hydrochloride. In some embodiments, the concentration of ethyl(methyl)amine hydrochloride is 10-250 mM. In some embodiments, the concentration of ethyl(methyl)amine hydrochloride is 50-110 mM.

In some embodiments, at least one amine of formula I comprises trimethylamine hydrochloride. In some embodiments, the concentration of trimethylamine hydrochloride is 10-250 mM. In some embodiments, the concentration of trimethylamine hydrochloride is 50-110 mM.

In some embodiments, the nucleic acid synthesis product yield is increased by at least 10%, 20%, 30%, 40%, 50%, 60%, 70%, 80%, 90%, 100%, 200%, 300%, 400%, or 500% as compared to the amount of product obtained in a reaction carried out under similar reaction conditions, but without amines. The amount of other salts may be reduced based on the presence of an amine in salt form.

In some embodiments, the tolerance to nucleic acid synthesis inhibitors is increased by at least 10%, 20%, 30%, 40%, 50%, 60%, 70%, 80%, 90%, 100%, 200%, 300%, 400%, or 500% as compared to the amount of product obtained in a reaction carried out under similar reaction conditions, but without amines and/or where the amount of other salts has been reduced based on the presence of an amine in salt form.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows amplification of a 727 base pair (bp) fragment from human genomic DNA by the thermophilic Taq DNA polymerase in the presence of 0 ng/μl, 19 ng/μl, 39 ng/μl, 77 ng/μl, or 154 ng/μl of xylan. Inhibitor concentration is indicated by the triangles at the top of the figure, with samples with increasing concentrations of xylan from 0-154 ng/μL loaded from left to right. PCR buffers contained 0 mM (Buffer 1), 10 mM (Buffer 2), 40 mM (Buffer 3), or 50 mM (Buffer 4) of diethylamine hydrochloride.

FIG. 2 shows amplification of a 727 bp fragment from human genomic DNA by the thermophilic Taq DNA polymerase in the presence of 0 ng/μl, 19 ng/μl, 39 ng/μl, 77 ng/μl, or 154 ng/μl of xylan. Inhibitor concentration is indicated by the triangles at the top of the figure, with samples with increasing concentrations of xylan from 0-154 ng/μL loaded from left to right. PCR buffers contained 0 mM (Buffer 1), 10 mM (Buffer 2), 40 mM (Buffer 3), or 50 mM (Buffer 4) of diisopropylamine hydrochloride.

FIG. 3 shows amplification of a 727 bp fragment from human genomic DNA by the thermophilic Taq DNA polymerase in the presence of 0 ng/μl, 19 ng/μl, 39 ng/μl, 77 ng/μl, or 154 ng/μl of xylan. Inhibitor concentration is indicated by the triangles at the top of the figure, with samples with increasing concentrations of xylan from 0-154 ng/μL loaded from left to right. PCR buffers contained 0 mM (Buffer 1), 10 mM (Buffer 2), 40 mM (Buffer 3), or 50 mM (Buffer 4) of ethyl(methyl)amine hydrochloride.

FIG. 4 shows amplification of a 727 bp fragment from human genomic DNA by the thermophilic Taq DNA polymerase in the presence of 0 ng/μl, 19 ng/μl, 39 ng/μl, 77 ng/μl, or 154 ng/μl of xylan. Inhibitor concentration is indicated by the triangles at the top of the figure, with samples with increasing concentrations of xylan from 0-154 ng/μL loaded from left to right. PCR buffers contained 0 mM (Buffer 1), 10 mM (Buffer 2), 40 mM (Buffer 3), or 50 mM (Buffer 4) of trimethylamine hydrochloride

FIG. 5 shows amplification of a 727 bp fragment from human genomic DNA by the thermophilic Taq DNA polymerase in the presence of 0 ng/μl, 19 ng/μl, 39 ng/μl, 77 ng/μl, or 154 ng/μl of xylan. Inhibitor concentration is indicated by the triangles at the top of the figure, with samples with increasing concentrations of xylan from 0-154 ng/μL loaded from left to right. PCR buffers contained 0 mM (Buffer 1), 10 mM (Buffer 2), 40 mM (Buffer 3), or 50 mM (Buffer 4) of dimethylamine hydrochloride.

FIG. 6 shows amplification of a 727 bp fragment from human genomic DNA by the thermophilic Taq DNA polymerase in the presence of 0 mM, 15 mM, 37 mM, 92 mM, or 230 mM of urea. Inhibitor concentration is indicated by the triangles at the top of the figure, with samples with increasing concentrations of urea from 0-230 mM loaded from left to right. PCR buffers contained 0 mM (Buffer 1), 10 mM (Buffer 2), 40 mM (Buffer 3), or 50 mM (Buffer 4) of diethylamine hydrochloride.

FIG. 7 shows amplification of a 727 bp fragment from human genomic DNA by the thermophilic Taq DNA polymerase in the presence of 0 mM, 15 mM, 37 mM, 92 mM, or 230 mM of urea. Inhibitor concentration is indicated by the triangles at the top of the figure, with samples with increasing concentrations of urea from 0-230 mM loaded from left to right. PCR buffers contained 0 mM (Buffer 1), 10 mM (Buffer 2), 40 mM (Buffer 3), or 50 mM (Buffer 4) of diisopropylamine hydrochloride.

FIG. 8 shows amplification of a 727 bp fragment from human genomic DNA by the thermophilic Taq DNA polymerase in the presence of 0 mM, 15 mM, 37 mM, 92 mM, or 230 mM of urea. Inhibitor concentration is indicated by the triangles at the top of the figure, with samples with increasing concentrations of urea from 0-230 mM loaded from left to right. PCR buffers contained 0 mM (Buffer 1), 10 mM (Buffer 2), 40 mM (Buffer 3), or 50 mM (Buffer 4) of ethyl(methyl)amine hydrochloride.

FIG. 9 shows amplification of a 727 bp fragment from human genomic DNA by the thermophilic Taq DNA polymerase in the presence of 0 mM, 15 mM, 37 mM, 92 mM, or 230 mM of urea. Inhibitor concentration is indicated by the triangles at the top of the figure, with samples with increasing concentrations of urea from 0-230 mM loaded from left to right. PCR buffers contained 0 mM (Buffer 1), 10 mM (Buffer 2), 40 mM (Buffer 3), or 50 mM (Buffer 4) of trimethylamine hydrochloride.

FIG. 10 shows amplification of a 727 bp fragment from human genomic DNA by the thermophilic Taq DNA polymerase in the presence of 0 mM, 15 mM, 37 mM, 92 mM, or 230 mM of urea. Inhibitor concentration is indicated by the triangles at the top of the figure, with samples with increasing concentrations of urea from 0-230 mM loaded from left to right. PCR buffers contained 0 mM (Buffer 1), 10 mM (Buffer 2), 40 mM (Buffer 3), or 50 mM (Buffer 4) of dimethylamine hydrochloride.

FIG. 11 shows amplification of a 727 bp fragment from human genomic DNA by the thermophilic Taq DNA polymerase in the presence of 0%, 0.02%, 0.04%, 0.05%, or 0.08% sodium citrate. Inhibitor concentration is indicated by the triangles at the top of the figure, with samples with increasing concentrations of sodium citrate from 0-0.08% loaded from left to right. PCR buffers contained 0 mM (Buffer 1), 10 mM (Buffer 2), 40 mM (Buffer 3), or 50 mM (Buffer 4) of diethylamine hydrochloride.

FIG. 12 shows amplification of a 727 bp fragment from human genomic DNA by the thermophilic Taq DNA polymerase in the presence of 0%, 0.02%, 0.04%, 0.05%, or 0.08% sodium citrate. Inhibitor concentration is indicated by the triangles at the top of the figure, with samples with increasing concentrations of sodium citrate from 0-0.08% loaded from left to right. PCR buffers contained 0 mM (Buffer 1), 10 mM (Buffer 2), 40 mM (Buffer 3), or 50 mM (Buffer 4) of diisopropylamine hydrochloride.

FIG. 13 shows amplification of a 727 bp fragment from human genomic DNA by the thermophilic Taq DNA polymerase in the presence of 0%, 0.02%, 0.04%, 0.05%, or 0.08% sodium citrate. Inhibitor concentration is indicated by the triangles at the top of the figure, with samples with increasing concentrations of sodium citrate from 0-0.08% loaded from left to right. PCR buffers contained 0 mM (Buffer 1), 10 mM (Buffer 2), 40 mM (Buffer 3), or 50 mM (Buffer 4) of ethyl(methyl)amine hydrochloride.

FIG. 14 shows amplification of a 727 bp fragment from human genomic DNA by the thermophilic Taq DNA polymerase in the presence of 0%, 0.02%, 0.04%, 0.05%, or 0.08% sodium citrate. Inhibitor concentration is indicated by the triangles at the top of the figure, with samples with increasing concentrations of sodium citrate from 0-0.08% loaded from left to right. PCR buffers contained 0 mM (Buffer 1), 10 mM (Buffer 2), 40 mM (Buffer 3), or 50 mM (Buffer 4) of trimethylamine hydrochloride.

FIG. 15 shows amplification of a 727 bp fragment from human genomic DNA by the thermophilic Taq DNA polymerase in the presence of 0%, 0.02%, 0.04%, 0.05%, or 0.08% sodium citrate. Inhibitor concentration is indicated by the triangles at the top of the figure, with samples with increasing concentrations of sodium citrate from 0-0.08% loaded from left to right. PCR buffers contained 0 mM (Buffer 1), 10 mM (Buffer 2), 40 mM (Buffer 3), or 50 mM (Buffer 4) of dimethylamine hydrochloride.

FIG. 16 shows amplification of a 1 kb fragment from human genomic DNA by the thermophilic Platinum SuperFi DNA polymerase in the presence of 0 M, 0.14 M, 0.28 M, 0.42 M, 0.56 M, 0.70 M, 0.84 M and 0.98 M urea. PCR buffers contained 110 mM KCl or 110 mM diethylamine hydrochloride.

FIG. 17 shows amplification of a 1 kb fragment from human genomic DNA by the thermophilic Platinum SuperFi DNA polymerase in the presence of 0 M, 0.14 M, 0.28 M, 0.42 M, 0.56 M, 0.70 M, 0.84 M and 0.98 M urea. PCR buffers contained 110 mM KCl, 110 mM ethyl(methyl)amine hydrochloride

FIG. 18 shows amplification of a 1 kb fragment from human genomic DNA by the thermophilic Platinum SuperFi DNA polymerase in the presence of 0 M, 0.14 M, 0.28 M, 0.42 M, 0.56 M, 0.70 M, 0.84 M and 0.98 M urea. PCR buffers contained 110 mM dimethylamine hydrochloride or 110 mM KCl in the control samples

FIG. 19 shows amplification of a 1 kb fragment from human genomic DNA by the thermophilic Platinum SuperFi DNA polymerase in the presence of 0 μl, 5 μl, 10 μl, 15 μl, 20 μl, 25 μl, 30 μl and 35 μl magnetic beads. PCR buffers contained 110 mM diethylamine hydrochloride or 110 mM KCl in the control samples.

FIG. 20 shows amplification of a 1 kb fragment from human genomic DNA by the thermophilic Platinum SuperFi DNA polymerase in the presence of 0 μl, 5 μl, 10 μl, 15 μl, 20 μl, 25 μl, 30 μl and 35 μl magnetic beads. PCR buffers contained 110 mM ethyl(methyl)amine hydrochloride or 110 mM KCl in the control samples.

FIG. 21 shows amplification of a 1 kb fragment from human genomic DNA by the thermophilic Platinum SuperFi DNA polymerase in the presence of 0 μl, 5 μl, 10 μl, 15 μl, 20 μl, 25 μl, 30 μl and 35 μl magnetic beads. PCR buffers contained 110 mM dimethylamine hydrochloride or 110 mM KCl in the control samples.

### DESCRIPTION OF THE SEQUENCES

The following table provides a listing of certain sequences referenced herein.

Description of the Sequences

| | | SEQ |
|---|---|---|
| | | ID |
| Description | Sequences | NO |
| Forward primer | GCCCTGTTCACCCGTTCTT | 1 |
| Reverse primer | GTGGCAACATGGCCCTTC | 2 |
| Forward primer | CAGGGCAGGGAGTTGAAGTT | 3 |
| Reverse primer | CAGGTTGGTATTGCCTTCTGG | 4 |

### DESCRIPTION OF THE EMBODIMENTS

### I. Definitions

As used herein, “amines” as used herein includes amines of Formula I:

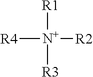

or salts thereof wherein

R1 is H; R2 is chosen from alkyl, alkenyl, alkynyl, or (CH2)n-R5, wherein n=1 to 3, and R5 is aryl, amino, thiol, mercaptan, phosphate, hydroxy, alkoxy; and R3 and R4 may be the same or different and are independently chosen from H or alkyl, with the proviso that if R2 is (CH2)n-R5, then at least one of R3 and/or R4 is alkyl. As such, amines include diethylamine hydrochloride, diisopropylamine hydrochloride, ethyl(methyl)amine hydrochloride, trimethylamine hydrochloride, and dimethylamine hydrochloride.

As used herein, “template” refers to any nucleic acid that can be used as the source material for nucleic acid synthesis. As such, a template may be present within a biological sample. As such, a template may be a synthetic/chemically synthesized nucleic acid. A template nucleic acid may be single stranded, double stranded or partially double stranded. Exemplary templates include RNA and DNA present in a biological sample or in any other sample that contains nucleic acid (e.g. a sample containing previously extracted, isolated or purified nucleic acids).

As used herein, “nucleic acid synthesis” refers to template-directed synthesis of a nucleic acid strand using a polymerase enzyme (i.e. an enzyme with polymerase activity). Nucleic acid synthesis includes all such template-directed nucleic acid synthesis by a polymerase, including, but not limited to, amplification, PCR, end point PCR (epPCR), real time or quantitative PCR (qPCR), one-step RT-PCR, sequencing, etc. An “application” of nucleic acid synthesis is any type of application, experiment, or procedure wherein nucleic acid synthesis is used. In some embodiments, nucleic acid synthesis is used to generate a nucleic acid that was based on or derived from a different template, such as generating DNA from an RNA template. In some embodiments, nucleic acid synthesis is used to generate a copy of a template that was present in a sample, which will be referred to as “amplifying” or “amplification.”

As used herein, an “nucleic acid synthesis inhibitor” refers to a compound or agent that inhibits or interferes with a reaction to synthesize nucleic acids. The nucleic acid synthesis inhibitor may be inherent in a sample from the original sample obtained. Exemplary original samples are organic and/or biological samples, such as blood, fabrics, tissues, feces, urine and/or soil. Nucleic acid synthesis inhibitors may be inherent in a sample from blood (e.g. heparin, hematin, EDTA, citrate, heme, heme-containing proteins), from soil or plant material (e.g. humic acid or plant polysaccharides such as xylan), from tissues (e.g. collagen), from urine (e.g. urea), from feces (e.g. bile salts, humic acid), etc. The nucleic acid synthesis inhibitor may also have been added to the sample in an upstream process or step (such as, e.g. nucleic acid extraction) before or during a reaction to synthesize nucleic acids. Exemplary nucleic acid synthesis inhibitors may be added from reagents used in the extraction and purification process (e.g. SDS, EDTA, ethanol, isopropanol, magnetic beads).

As used herein, a “microcarrier” also termed “microsphere”, “bead” relates to a particle of a size in the range of 0.5 μm to 100 μm. Preferably, the size is in the range of 1 μm to 50 μm, more preferably in the range of 1 μm to 25 μm, more preferably in the range of 1 μm to 10 μm. Microcarriers may be magnetic, i.e. comprise material that responds to a magnetic field, such as, but not limited to ferromagnetic, paramagnetic, and supermagnetic materials.

As used herein, a “stabilized formulation for long-term storage” refers to a formulation that maintains activity over long-term storage. Examples of a stabilized formulation for long-term storage include lyophilization or use of detergents in a composition. The stabilized formulation for long-term storage may provide for long-term storage of a composition in liquid form at 4° C. or at room temperature for one week, two weeks, one month or more than one month. The stabilized formulation for long-term storage may provide for long-term storage of a composition in liquid form at −20° C. for six months, one year, two years or more. Exemplary stabilized formulations may be compositions comprising glycerol or sucrose (saccharose) and/or detergents.

As used herein, “tolerance to inhibitors” refers to the ability of a polymerase to produce nucleic acid synthesis product in the presence of one or more nucleic acid synthesis inhibitors.

As used herein, “yield” refers to the amount of nucleic acid synthesis product produced by a polymerase.

As used herein, “thermophilic polymerase” or “thermophilic DNA polymerase” refers to a polymerase that has enhanced activity and/or stability at relatively high temperatures. Thermophilic nucleic acid polymerases typically have a temperature optimum of about 70-75° C. and may operate in a range of approximately 50° C. to 90° C. These enzymes are thermostable proteins. Thermostable proteins are typically stable up to a temperature of about 95° C.

As used herein, “non-thermophilic polymerase” or “non-thermophilic DNA polymerase” refers to a polymerase that has optimal activity at temperature lower than about 70-75° C.

As used herein, “hot start reaction” or “hot start PCR” refers to a protocol wherein an enzyme for a reaction is inactive until heated. A hot start protocol may reduce non-specific amplification and increase target yield and specificity by reducing binding of primers to non-specific templates and reducing formation of primer dimers.

### II. Compositions

Compositions comprising amines may improve nucleic acid synthesis product yield or tolerance to nucleic acid synthesis inhibitors by polymerases in nucleic acid synthesis. In some embodiments, the nucleic acid synthesis is for amplification of nucleic acid template.

A. Amine

The present composition comprises one or more amines of Formula I:

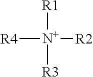

or salts thereof wherein R1 is H; R2 is chosen from alkyl, alkenyl, alkynyl, or (CH2)n-R5, wherein n=1 to 3, and R5 is aryl, amino, thiol, mercaptan, phosphate, hydroxy, alkoxy; and R3 and R4 may be the same or different and are independently chosen from H or alkyl, with the proviso that if R2 is (CH2)n-R5, then at least one of R3 and/or R4 is alkyl.

In some embodiments, R2 is an alkyl. In some embodiments, R2 is a C1-C5 alkyl. In some embodiments, the C1-C5 alkyl is linear. In some embodiments, the C1-C5 alkyl is branched. In some embodiments, R2 is a C1-C3 alkyl. In some embodiments, the alkyl is methyl.

In some embodiments, R3 and R4 are the same. In some embodiments, R3 and R4 are different. In some embodiments, R3 and/or R4 are H. In some embodiments, R3 and/or R4 are alkyl. R3 and/or R4 are a C1-C5 alkyl. In some embodiments, the C1-C5 alkyl is linear. In some embodiments, the C1-C5 alkyl is branched. In some embodiments, R3 and/or R4 are a C1-C3 alkyl. In some embodiments, the alkyl is methyl.

In some embodiments, when R2 is alkenyl or alkynyl, at least one of R3 and/or R4 is alkyl.

The amine may be a primary, secondary, or tertiary amine. In some embodiments the amine is monoalkylamine. In some embodiments the amine is dialkylamine. In some embodiments the amine is trialkylamine.

Table 1 presents some non-limiting examples of R1, R2, R3, and R4 groups of the invention.

TABLE 1
Non-limiting examples of R1, R2, R3,
and R4 groups of the invention

| R1 | R2 | R3 | R4 | |
|---|---|---|---|---|
| H | methyl | H | H | |
| H | Ethyl | H | H | |
| H | Propyl | H | H | |
| H | Butyl | H | H | |
| H | Pentyl | H | H | |
| H | methyl | methyl | H | and any |
| H | Ethyl | Ethyl | H | combinations |
| H | Propyl | Propyl | H | of R2 and R3 |
| H | Butyl | Butyl | H | |
| H | Pentyl | Pentyl | H | |
| H | methyl | methyl | methyl | and any |
| H | Ethyl | Ethyl | ethyl | combinations |
| H | Propyl | Propyl | propyl | of R2, R3 |
| H | Butyl | Butyl | butyl | and R4 |
| H | Pentyl | Pentyl | pentyl | |
| H | alkenyl, alkynyl, | methyl | H | and any |
| H | or (CH2)n—R5 | Ethyl | H | combinations |
| H | | Propyl | H | of R2 and R3 |
| H | | Butyl | H | |
| H | | Pentyl | H | |
| H | alkenyl, alkynyl, | methyl | methyl | and any |
| H | or (CH2)n—R5 | Ethyl | Ethyl | combinations |
| H | | Propyl | Propyl | of R2, R3 |
| H | | Butyl | Butyl | and R4 |
| H | | Pentyl | Pentyl | |

According to Table 1, the below listed amine salts are preferred.

Amine salts may be primary amine salts, with a total number of C atoms from 1 to 5 in the structure, regardless of atom arrangement (e.g. linear, branched, having different saturation degrees). In some examples, such amine salts do not comprise heteroatoms.

Exemplary primary amines are methylamine hydrochloride, ethylamine hydrochloride, propylamine hydrochloride, butylamine hydrochloride, pentylamine hydrochloride, propyl-2-amine hydrochloride, butyl-2-amine hydrochloride, pentyl-2-amine hydrochloride, pentyl-3-amine hydrochloride, ethenamine hydrochloride, 2-propen-1-amine hydrochloride, 1-propen-1-amine hydrochloride, 2-methyl-1-ethen-1-amine hydrochloride, 1-buten-1-amine hydrochloride, 2-buten-1-amine hydrochloride, 3-buten-1-amine hydrochloride, 2-methyl-2-propen-1-amine hydrochloride, 1-ethylethanamine hydrochloride, 2-penten-1-amine hydrochloride, 3-penten-1-amine hydrochloride, 4-penten-1-amine hydrochloride, 1-methyl-3-buten-1-amine hydrochloride, 2-methyl-3-buten-1-amine hydrochloride, 3-methyl-3-buten-1-amine hydrochloride, 1-methyl-2-buten-1-amine hydrochloride, 2-methyl-2-buten-1-amine hydrochloride, 3-methyl-2-buten-1-amine hydrochloride, 1-methyl-1-buten-1-amine hydrochloride, 2-methyl-1-buten-1-amine hydrochloride, 3-methyl-1-buten-1-amine hydrochloride, 1-ethyl-1-propen-1-amine hydrochloride, 1-ethyl-2-propen-1-amine hydrochloride.

Amine salts may be secondary amine salts with a total number of C atoms being from 2 to 15. Secondary amine salts may have a total number of C atoms from 2 to 10. Further, the total number of C atoms in a secondary amine may be from 2 to 6. Secondary amine salts may have a total number of C atoms from 2 to 4. In some examples, such amine salts do not comprise heteroatoms.

Exemplary secondary amines are N-methylmethanamine hydrochloride (dimethylamine hydrochloride), N-methylethan-1-amine hydrochloride (ethyl(methyl)amine hydrochloride), N-methylpropan-1-amine hydrochloride, N-metylbutan-1-amine hydrochloride, N-methylpentan-1-amine hydrochloride, N-ethylethan-1-amine hydrochloride (diethylamine hydrochloride), N-ethylpropan-1-amine hydrochloride, N-ethylbutan-1-amine hydrochloride, N-ethylpentan-1-amine hydrochloride, N-propylpropan-1-amine hydrochloride, N-propylbutan-1-amine hydrochloride, N-propylpentan-1-amine hydrochloride, N-butylbutan-1-amine hydrochloride, N-butylpentan-1-amine hydrochloride, N-pentylpentan-1-amine hydrochloride, N-methylpropan-2-amine hydrochloride, N-metylbutan-2-amine hydrochloride, N-methylpentan-2-amine hydrochloride, N-methylpentan-3-amine hydrochloride, N-ethylpropan-2-amine hydrochloride, N-ethylbutan-2-amine hydrochloride, N-ethylpentan-2-amine hydrochloride, N-ethylpentan-3-amine hydrochloride, N-propylpropan-2-amine hydrochloride, N-2-propylpropan-2-amine hydrochloride (diisopropylamine hydrochloride), N-propylbutan-2-amine hydrochloride, N-2-propylbutan-1-amine hydrochloride, N-2-propylbutan-2-amine hydrochloride, N-propylpentan-2-amine hydrochloride, N-propylpentan-3-amine hydrochloride, N-2-propylpentan-1-amine hydrochloride, N-2-propylpentan-2-amine hydrochloride, N-2-propylpentan-3-amine hydrochloride, N-butylbutan-2-amine hydrochloride, N-2-butylbutan-2-amine hydrochloride, N-2-butylpentan-1-amine hydrochloride, N-butylpentan-2-amine hydrochloride, N-butylpentan-3-amine hydrochloride, N-pentylpentan-2-amine hydrochloride, N-pentylpentan-3-amine hydrochloride, N-2-pentylpentan-2-amine hydrochloride, N-3-pentylpentan-3-amine hydrochloride, N-2-pentylpentan-3-amine hydrochloride, N-methylethenamine hydrochloride, N-ethylethenamine hydrochloride, N-propylethenamine hydrochloride, N-butylethenamine hydrochloride, N-pentylethenamine hydrochloride, N-mehyl-2-propen-1-amine hydrochloride, N-methyl-1-propen-1-amine hydrochloride, N-methyl-2-methyl-1-ethen-1-amine hydrochloride, N-ehyl-2-propen-1-amine hydrochloride, N-ethyl-1-propen-1-amine hydrochloride, N-ethyl-2-methyl-1-ethen-1-amine hydrochloride, N-propyl-2-propen-1-amine hydrochloride, N-propyl-1-propen-1-amine hydrochloride, N-propyl-2-methyl-1-ethen-1-amine hydrochloride, N-butyl-2-propen-1-amine hydrochloride, N-butyl-1-propen-1-amine hydrochloride, N-butyl-2-methyl-1-ethen-1-amine hydrochloride, N-pentyl-2-propen-1-amine hydrochloride, N-pentyl-1-propen-1-amine hydrochloride, N-pentyl-2-methyl-1-ethen-1-amine hydrochloride, N-methyl-1-buten-1-amine hydrochloride, N-methyl-2-buten-1-amine hydrochloride, N-methyl-3-buten-1-amine hydrochloride, N-methyl-2-methyl-2-propen-1-amine hydrochloride, N-methyl-1-ethylethanamine hydrochloride, N-ethyl-1-buten-1-amine hydrochloride, N-ethyl-2-buten-1-amine hydrochloride, N-ethyl-3-buten-1-amine hydrochloride, N-ethyl-2-methyl-2-propen-1-amine hydrochloride, N-ethyl-1-ethylethanamine hydrochloride, N-propyl-1-buten-1-amine hydrochloride, N-propyl-2-buten-1-amine hydrochloride, N-propyl-3-buten-1-amine hydrochloride, N-propyl-2-methyl-2-propen-1-amine hydrochloride, N-propyl-1-ethylethanamine hydrochloride, N-butyl-1-buten-1-amine hydrochloride, N-b 1-2-buten-1-amine hydrochloride, N-butyl-3-buten-1-amine hydrochloride, N-butyl-2-methyl-2-propen-1-amine hydrochloride, N-butyl-1-ethylethanamine hydrochloride, N-pentyl-1-buten-1-amine hydrochloride, N-pentyl-2-buten-1-amine hydrochloride, N-pentyl-3-buten-1-amine hydrochloride, N-pentyl-2-methyl-2-propen-1-amine hydrochloride, N-pentyl-1-ethylethanamine hydrochloride, N-methyl-2-penten-1-amine hydrochloride, N-methyl-3-penten-1-amine hydrochloride, N-methyl-4-penten-1-amine hydrochloride, N-methyl-1-methyl-3-buten-1-amine hydrochloride, N-methyl-2-methyl-3-buten-1-amine hydrochloride, N-methyl-3-methyl-3-buten-1-amine hydrochloride, N-methyl-1-methyl-2-buten-1-amine hydrochloride, N-methyl-2-methyl-2-buten-1-amine hydrochloride, N-methyl-3-methyl-2-buten-1-amine hydrochloride, N-methyl-1-methyl-1-buten-1-amine hydrochloride, N-methyl-2-methyl-1-buten-1-amine hydrochloride, N-methyl-3-methyl-1-buten-1-amine hydrochloride, N-methyl-1-ethyl-1-propen-1-amine hydrochloride, N-methyl-1-ethyl-2-propen-1-amine hydrochloride, N-ethyl-2-penten-1-amine hydrochloride, N-ethyl-3-penten-1-amine hydrochloride, N-ethyl-4-penten-1-amine hydrochloride, N-ethyl-1-methyl-3-buten-1-amine hydrochloride, N-ethyl-2-methyl-3-buten-1-amine hydrochloride, N-ethyl-3-methyl-3-buten-1-amine hydrochloride, N-ethyl-1-methyl-2-buten-1-amine hydrochloride, N-ethyl-2-methyl-2-buten-1-amine hydrochloride, N-ethyl-3-methyl-2-buten-1-amine hydrochloride, N-ethyl-1-methyl-1-buten-1-amine hydrochloride, N-ethyl-2-methyl-1-buten-1-amine hydrochloride, N-ethyl-3-methyl-1-buten-1-amine hydrochloride, N-ethyl-1-ethyl-1-propen-1-amine hydrochloride, N-ethyl-1-ethyl-2-propen-1-amine hydrochloride, N-propyl-2-penten-1-amine hydrochloride, N-propyl-3-penten-1-amine hydrochloride, N-propyl-4-penten-1-amine hydrochloride, N-propyl-1-methyl-3-buten-1-amine hydrochloride, N-propyl-2-methyl-3-buten-1-amine hydrochloride, N-propyl-3-methyl-3-buten-1-amine hydrochloride, N-propyl-1-methyl-2-buten-1-amine hydrochloride, N-propyl-2-methyl-2-buten-1-amine hydrochloride, N-propyl-3-methyl-2-buten-1-amine hydrochloride, N-propyl-1-methyl-1-buten-1-amine hydrochloride, N-propyl-2-methyl-1-buten-1-amine hydrochloride, N-propyl-3-methyl-1-buten-1-amine hydrochloride, N-propyl-1-ethyl-1-propen-1-amine hydrochloride, N-propyl-1-ethyl-2-propen-1-amine hydrochloride, N-butyl-2-penten-1-amine hydrochloride, N-butyl-3-penten-1-amine hydrochloride, N-butyl-4-penten-1-amine hydrochloride, N-butyl-1-methyl-3-buten-1-amine hydrochloride, N-butyl-2-methyl-3-buten-1-amine hydrochloride, N-butyl-3-methyl-3-buten-1-amine hydrochloride, N-butyl-1-methyl-2-buten-1-amine hydrochloride, N-butyl-2-methyl-2-buten-1-amine hydrochloride, N-butyl-3-methyl-2-buten-1-amine hydrochloride, N-butyl-1-methyl-1-buten-1-amine hydrochloride, N-butyl-2-methyl-1-buten-1-amine hydrochloride, N-butyl-3-methyl-1-buten-1-amine hydrochloride, N-butyl-1-ethyl-1-propen-1-amine hydrochloride, N-butyl-1-ethyl-2-propen-1-amine hydrochloride, N-pentyl-2-penten-1-amine hydrochloride, N-pentyl-3-penten-1-amine hydrochloride, N-pentyl-4-penten-1-amine hydrochloride, N-pentyl-1-methyl-3-buten-1-amine hydrochloride, N-pentyl-2-methyl-3-buten-1-amine hydrochloride, N-pentyl-3-methyl-3-buten-1-amine hydrochloride, N-pentyl-1-methyl-2-buten-1-amine hydrochloride, N-pentyl-2-methyl-2-buten-1-amine hydrochloride, N-pentyl-3-methyl-2-buten-1-amine hydrochloride, N-pentyl-1-methyl-1-buten-1-amine hydrochloride, N-pentyl-2-methyl-1-buten-1-amine hydrochloride, N-pentyl-3-methyl-1-buten-1-amine hydrochloride, N-pentyl-1-ethyl-1-propen-1-amine hydrochloride, N-pentyl-1-ethyl-2-propen-1-amine hydrochloride.

More preferred examples are N-methylmethanamine hydrochloride (dimethylamine hydrochloride), N-methylethan-1-amine hydrochloride (ethyl(methyl)amine hydrochloride), N-methylpropan-1-amine hydrochloride, N-ethylethan-1-amine hydrochloride (diethylamine hydrochloride), N-ethylpropan-1-amine hydrochloride, N-2-propylpropan-2-amine hydrochloride (diisopropylamine hydrochloride),

Amine salts may be tertiary amine salts with a total number of C atoms being from 3 to 15. Tertiary amine salts may have a total number of C atoms from 3 to 9, wherein any of R2, R3 and R4 comprise not more than 5 C atoms. Further, the total number of C atoms in a tertiary amine may be from 3 to 6 . . . . In some examples, such amine salts do not comprise heteroatoms.

Exemplary tertiary amines are N,N-dimethylmethanamine hydrochloride (trimethylamine hydrochloride), N,N-dimethylethan-1-amine hydrochloride, N,N-dimethylpropan-1-amine hydrochloride, N,N-dimethylbutan-1-amine hydrochloride, N,N-dimethylpentan-1-amine hydrochloride, N-ethyl-N-methylmethanamine hydrochloride, N-ethyl-N-methylpropan-1-amine hydrochloride, N-ethyl-N-methylbutan-1-amine hydrochloride, N-ethyl-N-methylpentane-1-amine hydrochloride, N-methyl-N-propylpropan-1-amine hydrochloride, N-methyl-N-propylbutan-1-amine hydrochloride, N-methyl-N-propylpentan-1-amine hydrochloride, N-butyl-N-methylbutan-1-amine hydrochloride, N-butyl-N-methylpentan-1-amine hydrochloride, N,N-diethylethanamine hydrochloride, N,N-diethylpropan-1-amine hydrochloride, N,N-diethylbutan-1-amine hydrochloride, N,N-diethylpentan-1-amine hydrochloride, N-ethyl-N-propylpropan-1-amine hydrochloride, N-ethyl-N-propylbutan-1-amine hydrochloride, N-ethyl-N-propylpentan-1-amine hydrochloride, N-ethyl-N-butylbutan-1-amine hydrochloride, N-butyl-N-ethylpentan-amine hydrochloride, N-ethyl-N-pentylpentan-1-amine hydrochloride, N,N-dipropylpropan-1-amine hydrochloride, N,N-dipropylbutan-1-amine hydrochloride, N,N-dipropylpenta-1-amine hydrochloride, N-butyl-N-propylbutan-1-amine hydrochloride, N,N-dibutylbutan-1-amine hydrochloride, N,N-dibutylpentan-1-amine hydrochloride, N-methyl-N-pentylpentan-1-amine hydrochloride, N-ethyl-N-pentylpentan-1-amine hydrochloride, N-propyl-N-pentylpentan-1-amine hydrochloride, N-butyl-N-pentylpentan-1-amine hydrochloride, N,N-dipentylpentan-1-amine hydrochloride,N,N-dimethylethenamine hydrochloride, N,N-diethylethenamine hydrochloride, N,N-dipropylethenamine hydrochloride, N,N-dibutylethenamine hydrochloride, N,N-dipentylethenamine hydrochloride, N,N-dimehyl-2-propen-1-amine hydrochloride, N,N-dimehyl-1-propen-1-amine hydrochloride, N,N-dimehyl-2-methyl-1-ethen-1-amine hydrochloride, N,N-diehyl-2-propen-1-amine hydrochloride, N,N-diehyl-1-propen-1-amine hydrochloride, N,N-diethyl-2-methyl-1-ethen-1-amine hydrochloride, N,N-dipropyl-2-propen-1-amine hydrochloride, N,N-dipropyl-1-propen-1-amine hydrochloride, N,N-dipropyl-2-methyl-1-ethen-1-amine hydrochloride, N,N-dibutyl-2-propen-1-amine hydrochloride, N,N-dibutyl-1-propen-1-amine hydrochloride, N,N-dibutyl-2-methyl-1-ethen-1-amine hydrochloride, N,N-dipentyl-2-propen-1-amine hydrochloride, N,N-dipentyl-1-propen-1-amine hydrochloride, N,N-dipentyl-2-methyl-1-ethen-1-amine hydrochloride, N,N-dimethyl-1-buten-1-amine hydrochloride, N,N-dimethyl-2-buten-1-amine hydrochloride, N,N-dimethyl-3-buten-1-amine hydrochloride, N,N-dimethyl-2-methyl-2-propen-1-amine hydrochloride, N,N-dimethyl-1-ethylethanamine hydrochloride, N,N-diethyl-1-buten-1-amine hydrochloride, N,N-diethyl-2-buten-1-amine hydrochloride, N,N-diethyl-3-buten-1-amine hydrochloride, N,N-diethyl-2-methyl-2-propen-1-amine hydrochloride, N,N-diethyl-1-ethylethanamine hydrochloride, N,N-dipropyl-1-buten-1-amine hydrochloride, N,N-dipropyl-2-buten-1-amine hydrochloride, N,N-dipropyl-3-buten-1-amine hydrochloride, N,N-dipropyl-2-methyl-2-propen-1-amine hydrochloride, N,N-dipropyl-1-ethylethanamine hydrochloride, N,N-dibutyl-1-buten-1-amine hydrochloride, N,N-dibutyl-2-buten-1-amine hydrochloride, N,N-dibutyl-3-buten-1-amine hydrochloride, N,N-dibutyl-2-methyl-2-propen-1-amine hydrochloride, N,N-dibutyl-1-ethylethanamine hydrochloride, N,N-dipentyl-1-buten-1-amine hydrochloride, N,N-dipentyl-2-buten-1-amine hydrochloride, N,N-dipentyl-3-buten-1-amine hydrochloride, N,N-dipentyl-2-methyl-2-propen-1-amine hydrochloride, N,N-dipentyl-1-ethylethanamine hydrochloride, N,N-dimethyl-2-penten-1-amine hydrochloride, N,N-dimethyl-3-penten-1-amine hydrochloride, N,N-dimethyl-4-penten-1-amine hydrochloride, N,N-dimethyl-1-methyl-3-buten-1-amine hydrochloride, N,N-dimethyl-2-methyl-3-buten-1-amine hydrochloride, N,N-dimethyl-3-methyl-3-buten-1-amine hydrochloride, N,N-dimethyl-1-methyl-2-buten-1-amine hydrochloride, N,N-dimethyl-2-methyl-2-buten-1-amine hydrochloride, N,N-dimethyl-3-methyl-2-buten-1-amine hydrochloride, N,N-dimethyl-1-methyl-1-buten-1-amine hydrochloride, N,N-dimethyl-2-methyl-1-buten-1-amine hydrochloride, N,N-dimethyl-3-methyl-1-buten-1-amine hydrochloride, N,N-dimethyl-1-ethyl-1-propen-1-amine hydrochloride, N,N-dimethyl-1-ethyl-2-propen-1-amine hydrochloride, N,N-diethyl-2-penten-1-amine hydrochloride, N,N-diethyl-3-penten-1-amine hydrochloride, N,N-diethyl-4-penten-1-amine hydrochloride, N,N-diethyl-1-methyl-3-buten-1-amine hydrochloride, N,N-diethyl-2-methyl-3-buten-1-amine hydrochloride, N,N-diethyl-3-methyl-3-buten-1-amine hydrochloride, N,N-diethyl-1-methyl-2-buten-1-amine hydrochloride, N,N-diethyl-2-methyl-2-buten-1-amine hydrochloride, N,N-diethyl-3-methyl-2-buten-1-amine hydrochloride, N,N-diethyl-1-methyl-1-buten-1-amine hydrochloride, N,N-diethyl-2-methyl-1-buten-1-amine hydrochloride, N,N-diethyl-3-methyl-1-buten-1-amine hydrochloride, N,N-diethyl-1-ethyl-1-propen-1-amine hydrochloride, N,N-diethyl-1-ethyl-2-propen-1-amine hydrochloride, N,N-dipropyl-2-penten-1-amine hydrochloride, N,N-dipropyl-3-penten-1-amine hydrochloride, N,N-dipropyl-4-penten-1-amine hydrochloride, N,N-dipropyl-1-methyl-3-buten-1-amine hydrochloride, N,N-dipropyl-2-methyl-3-buten-1-amine hydrochloride, N,N-dipropyl-3-methyl-3-buten-1-amine hydrochloride, N,N-dipropyl-1-methyl-2-buten-1-amine hydrochloride, N,N-dipropyl-2-methyl-2-buten-1-amine hydrochloride, N,N-dipropyl-3-methyl-2-buten-1-amine hydrochloride, N,N-dipropyl-1-methyl-1-buten-1-amine hydrochloride, N,N-dipropyl-2-methyl-1-buten-1-amine hydrochloride, N,N-dipropyl-3-methyl-1-buten-1-amine hydrochloride, N,N-dipropyl-1-ethyl-1-propen-1-amine hydrochloride, N,N-dipropyl-1-ethyl-2-propen-1-amine hydrochloride, N,N-dibutyl-2-penten-1-amine hydrochloride, N,N-dibutyl-3-penten-1-amine hydrochloride, N,N-dibutyl-4-penten-1-amine hydrochloride, N,N-dibutyl-1-methyl-3-buten-1-amine hydrochloride, N,N-dibutyl-2-methyl-3-buten-1-amine hydrochloride, N,N-dibutyl-3-methyl-3-buten-1-amine hydrochloride, N,N-dibutyl-1-methyl-2-buten-1-amine hydrochloride, N,N-dibutyl-2-methyl-2-buten-1-amine hydrochloride, N,N-dibutyl-3-methyl-2-buten-1-amine hydrochloride, N,N-dibutyl-1-methyl-1-buten-1-amine hydrochloride, N,N-dibutyl-2-methyl-1-buten-1-amine hydrochloride, N,N-dibutyl-3-methyl-1-buten-1-amine hydrochloride, N,N-dibutyl-1-ethyl-1-propen-1-amine hydrochloride, N,N-dibutyl-1-ethyl-2-propen-1-amine hydrochloride, N,N-dipentyl-2-penten-1-amine hydrochloride, N,N-dipentyl-3-penten-1-amine hydrochloride, N,N-dipentyl-4-penten-1-amine hydrochloride, N,N-dipentyl-1-methyl-3-buten-1-amine hydrochloride, N,N-dipentyl-2-methyl-3-buten-1-amine hydrochloride, N,N-dipentyl-3-methyl-3-buten-1-amine hydrochloride, N,N-dipentyl-1-methyl-2-buten-1-amine hydrochloride, N,N-dipentyl-2-methyl-2-buten-1-amine hydrochloride, N,N-dipentyl-3-methyl-2-buten-1-amine hydrochloride, N,N-dipentyl-1-methyl-1-buten-1-amine hydrochloride, N,N-dipentyl-2-methyl-1-buten-1-amine hydrochloride, N,N-dipentyl-3-methyl-1-buten-1-amine hydrochloride, N,N-dipentyl-1-ethyl-1-propen-1-amine hydrochloride, N,N-dipentyl-1-ethyl-2-propen-1-amine hydrochloride.

More preferred examples are N,N-dimethylmethanamine hydrochloride (trimethylamine hydrochloride), N,N-dimethylethan-1-amine hydrochloride, N,N-dimethylpropan-1-amine hydrochloride, N-ethyl-N-methylmethanamine hydrochloride.

In other examples, other salts of listed amines may be used, such as chloride, sulfate, or acetate salt.

In some embodiments, the amine is in a salt form. In some embodiments, the salt is any salt compatible with an enzymatic nucleic acid synthesis reaction. In some embodiments, the salt is an anion. In some embodiments, the salt is a chloride, sulfate, or acetate salt.

In some embodiments, a composition comprises one amine. In some embodiments, a composition comprises two different amines. In some embodiments, a composition comprises three different amines. In some embodiments, a composition comprises four different amines.

In some embodiments, the total amount of amine(s) in a composition is about 10-250 mM. In some embodiments, the total amount of amine(s) in a composition is about 50-110 mM. In some embodiments, the total amount of amine(s) in a composition is less than about 250 mM. In some embodiments, the total amount of amine(s) in a composition is at least about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM.

In some embodiments, one of the amines comprises dimethylamine hydrochloride. In some embodiments, the concentration of dimethylamine hydrochloride is about 10-250 mM. In some embodiments, the concentration of dimethylamine hydrochloride is about 50-110 mM.

In some embodiments, one of the amines comprises diethylamine hydrochloride. In some embodiments, the concentration of diethylamine hydrochloride is about 10-250 mM. In some embodiments, the concentration of diethylamine hydrochloride is about 50-110 mM.

In some embodiments, one of the amines comprises diisopropylamine hydrochloride. In some embodiments, the concentration of diisopropylamine hydrochloride is about 10-250 mM. In some embodiments, the concentration of diisopropylamine hydrochloride is about 50-110 mM.

In some embodiments, one of the amines comprises ethyl(methyl)amine hydrochloride. In some embodiments, the concentration of ethyl(methyl)amine hydrochloride is about 10-250 mM. In some embodiments, the concentration of ethyl(methyl)amine hydrochloride is about 50-110 mM.

In some embodiments, one of the amines comprises trimethylamine hydrochloride. In some embodiments, the concentration of trimethylamine hydrochloride is about 10-250 mM. In some embodiments, the concentration of trimethylamine hydrochloride is about 50-110 mM.

B. Nucleic Acid Synthesis Inhibitors

Nucleic acid synthesis inhibitors may include contaminants that are inherent in the samples or agents added to the sample in upstream processes. Amines may improve nucleic acid synthesis product yield or tolerance to nucleic acid synthesis inhibitors in nucleic acid synthesis reactions.

In some embodiments, nucleic acid synthesis inhibitors decrease PCR yield. In some embodiments, nucleic acid synthesis inhibitors abolish amplification.

In some embodiments, nucleic acid synthesis inhibitors are present in the biological sample. In some embodiments, the nucleic acid synthesis inhibitor is present in the biological sample such as blood, serum, plasma, urine, fabrics, tissues, or soil.

In some embodiments, nucleic acid synthesis inhibitors are present in a sample containing nucleic acids, e.g. previously extracted, isolated or purified nucleic acids.

In some embodiments, nucleic acid synthesis inhibitors are purposely added to the sample. In some embodiments, nucleic acid synthesis inhibitors are added as a result of the sample processing and nucleic acid extraction steps (see Schrader et al., Journal of Applied Microbiology 113:1014-1026 (2012) and Alaeddini Forensic Science International: Genetics 6:297-305 (2012)).

In some embodiments, the nucleic acid synthesis inhibitor is a polyanion. In some embodiments, the polyanion is heparin or xylan.

In some embodiments, the nucleic acid synthesis inhibitor is a chaotropic agent. In some embodiments, the chaotropic agent is sodium dodecyl sulfate or urea.

In some embodiments, the nucleic acid synthesis inhibitor is a metal ion. In some embodiments, the metal ion is calcium.

In some embodiments, the nucleic acid synthesis inhibitor is a protein. In some embodiments, the nucleic acid synthesis inhibitor is collagen, heme or heme-containing proteins.

In some embodiments, the nucleic acid synthesis inhibitor is an organic compound. In some embodiments, the inhibitor is humic acid, or bile salts.

In some embodiments, the nucleic acid synthesis inhibitor is a chelator. In some embodiments, the chelator is citrate or EDTA.

In some embodiments, the nucleic acid synthesis inhibitor is an organic solvent. In some embodiments, the organic solvent is ethanol or isopropanol.

In some embodiments, the nucleic acid synthesis inhibitor is a nucleic acid intercalating dye.

In some embodiments, the nucleic acid synthesis reaction is inhibited by the presence of magnetic beads.

1. Magnetic Beads

Magnetic beads are widely used in biochemical reactions, as they provide an excellent solid support for a wide range of biomagnetic separations, molecular manipulations, and affinity isolations. Magnetic beads can be used in nucleic acid separation/purification methods (e.g. the nucleic acids are bound to magnetic beads under one conditions, and released under other conditions). However, the nucleic acid synthesis inhibition can be observed in the presence of magnetic beads.

In some embodiments, the magnetic beads are carboxylated magnetic beads. In some embodiments, the magnetic beads are Agencourt® AMPure® XP (Beckman Coulter, Inc), Sera-Mag™ SpeedBeads™ (GE Healthcare Life Sciences), MyOne carboxylated beads (DynaBeads), or Mag-Bind® RXNPure (Omega Bio-tek, Inc).

In some embodiments, nucleic acid is non-sequence specifically bound to magnetic beads. In some embodiments, nucleic acid is reversibly bound to magnetic beads. In some embodiments, nucleic acid is non-sequence specifically reversibly bound to magnetic beads. In some embodiments, nucleic acid is bound to magnetic beads in sequence-specific manner. In some embodiments, nucleic acid is non-reversibly bound to magnetic beads.

In some embodiments, magnetic beads are carried over or included in a nucleic acid synthesis step. In some embodiments, inhibition by magnetic beads is concentration-dependent.

In some embodiments, magnetic beads are intentionally left in a sample. In some embodiments, magnetic beads are intentionally left in a sample to reduce the number of upstream processing steps. In some embodiments, magnetic beads are intentionally left in a sample to reduce time needed to process a sample. In some embodiments, nucleic acids (e.g. nucleic acid template or primers) are bound or immobilized on magnetic beads. In some embodiments, nucleic acids (e.g. nucleic acid template or primers) are bound or immobilized on magnetic beads and magnetic beads are intentionally left in a sample.

In some embodiments, traces of magnetic beads remain after a step to remove the beads.

C. Types of Compositions

Kits or compositions claimed herein may further comprise additional components, including an enzyme for nucleic acid synthesis. In some embodiments, the amine may be supplied separately from a polymerase. In some embodiments, an amine may be supplied together with an enzyme, which may be referred to as a “MasterMix.” In some embodiments, a MasterMix comprises a polymerase. In some embodiments, the one or more amine and one or more enzymes for synthesizing nucleic acid molecules are comprised in a single container.

In some embodiments, the amine is supplied in a separate container from the enzyme and other components. In some embodiments, the amine is not supplied in a reaction buffer. In some embodiments, the amine is provided as an aqueous solution comprising the amine or a salt thereof.

In some embodiments, the amine is supplied together with enzyme and other components. In some embodiments, the amine is supplied in a reaction buffer.

In some embodiments, the reaction buffer may comprise additional components. These additional components may be items necessary for the synthesis of a nucleic acid molecule (such as dNTPs or NTPs) or reagents to improve performance or storage of a reaction solution (such as a protein stabilizer or preservative).

In some embodiments, the composition is provided at 2×, 5×, 10×, or greater concentration. If a composition is provided at 2×, for example, the concentrations discussed herein are multiplied (e.g., as noted above; doubled for 2×). A 2× reaction composition is typically diluted by 2-fold, for example, when the template nucleic acid and/or primers are added to the composition.

In some embodiments, components necessary for the synthesis of a nucleic acid molecule include an enzyme, nucleic acid molecules, dNTPs or NTPs, buffer, and cofactor. In some embodiments, in addition to an enzyme the composition comprises one or more additional components chosen from (i) one or more nucleic acid molecules; (ii) one or more nucleotides; (iii) one or more buffering salts; and (iv) one or more cofactors.

1. Enzymes

In some embodiments, an enzyme of the invention comprises a polymerase. In some embodiments, the polymerase of the invention may be a thermophilic polymerase. In some embodiments, the polymerase of the invention may be a non-thermophilic polymerase.

In some embodiments, the polymerase comprises a DNA polymerase. In some embodiments, the DNA polymerase comprises Phi29 or its derivatives (e.g. U.S. Pat. No. 9,422,535B2), Bsm, Bst, T4, T7, DNA Pol I, or Klenow Fragment, and/or mutants, variants and derivatives thereof.

In some embodiments, the DNA polymerase comprises a thermophilic DNA polymerase. In some embodiments, the thermophilic DNA polymerase comprises Taq, Tbr, Tfl, Tth, Tli, Tfi, Tne, Tma, Pfu, Pwo, Kod, VENT™, DEEPVENT™, DNA polymerase; Phusion DNA polymerase; Phusion U DNA polymerase; SuperFi DNA polymerase; SuperFi U DNA Polymerase; and/or mutants, variants and derivatives thereof (see e.g. US20170204384A1, U.S. Pat. No. 9,493,848B2, U.S. Pat. No. 6,627,424B1, 62/524,730); and/or GoTaq G2 Hot Start Polymerase (Promega), OneTaq® Hot Start DNA Polymerase (NEB), TaKaRa Taq™ DNA Polymerase Hot Start (TaKaRa), KAPA2G Robust HotStart DNA Polymerase (KAPA), FastStart Taq DNA Polymerase (Roche), HotStart Taq DNA Polymerase (Qiagen), Q5 DNA Polymerase, Kapa HiFi DNA Polymerase, PrimeStar Max DNA Polymerase, PrimeStar GXL DNA Polymerase.

In some embodiments, the DNA polymerase comprises a non-thermophilic DNA polymerase.

In some embodiments, the DNA polymerase comprises a chimeric DNA polymerase. In some embodiments, the chimeric DNA polymerase comprises a sequence nonspecific double-stranded DNA (dsDNA) binding domain Exemplary DNA binding domains include Sso7d from Sulfolobus solfataricus, Sac7d, Sac7a, Sac7b, and Sac7e from S. acidocaldarius, and Ssh7a and Ssh7b from Sulfolobus shibatae, Pae3192, Pae0384, and Ape3192, HMf family archaeal histone domains, and archaeal PCNA homolog.

In some embodiments, the polymerase comprises an RNA polymerase. In some embodiments, the RNA polymerase comprises SP6, T7, or T3 RNA polymerase and mutants, variants, and derivatives thereof.

In some embodiments, the polymerase comprises an RNA-dependent DNA polymerase. In some embodiments, the RNA-dependent DNA polymerase comprises a reverse transcriptase (RT). In some embodiments, the reverse transcriptase comprises M-MLV reverse transcriptase, RSV reverse transcriptase, AMV reverse transcriptase, RAV reverse transcriptase, MAV reverse transcriptase, or HIV reverse transcriptase and/or mutants, variants, and derivatives thereof (see e.g. U.S. Pat. Nos. 8,835,148, 7,056,716, 7,078,208); and/or SuperScript II reverse transcriptase, SuperScript III reverse transcriptase, SuperScript IV reverse transcriptase, Maxima reverse transcriptase, GoScript reverse transcriptase, PrimeScript reverse transcriptase, iScript reverse transcriptase, Sensiscript reverse transcriptase, ProtoScript reverse transcriptase, AffinityScript Reverse Transcriptase, NxtScript Reverse Transcriptase, RnaUsScript Reverse Transcriptase, RocketScript Reverse Transcriptase, GoScript Reverse Transcriptase, and/or Thermoscript reverse transcriptase.

In some embodiments, reverse transcriptases have reduced or substantially reduced RNase H activity.

2. Additional Components

In some embodiments, in addition to the amine (and optionally the enzyme) components, the present compositions comprise one or more buffers and/or cofactors necessary for synthesis of a nucleic acid molecule.

In some embodiments, the one or more nucleic acid molecules comprise RNA or DNA. In some embodiments, the one or more nucleic acid molecules comprise at least one primer.

In some embodiments, the one or more nucleotides comprise dNTPs or NTPs.

In some embodiments, buffers for use in forming the present compositions comprises acetate, sulfate, hydrochloride, phosphate or free acid forms of Tris-(hydroxymethyl)aminomethane (TRIS®). In some embodiments, alternative buffers of the same approximate ionic strength and pKa as TRIS® may be used.

In some embodiments, in addition to the buffer salts, cofactor salts such as magnesium (such as magnesium chloride, magnesium sulfate or magnesium acetate) are included in the compositions.

In some embodiments, additional salts of potassium are added. In some embodiments, the additional salts of potassium comprises potassium chloride or potassium acetate. In some embodiments, the potassium chloride concentration may be reduced or potassium chloride may be omitted based on the presence of an amine.

In some embodiments, the amount of other salts in a composition may be reduced based on the presence of an amine in salt form. In some embodiments, the amount of other salts may be reduced by, e.g. at least 5%, at least 10%, at least 20%, at least 80% or by more than 90%. In some embodiments, the amine in salt form may be in addition to other salts in a composition.

In some embodiments, KCl can be absent in the composition. In some embodiments, KCl is absent if at least one of the amines of the kit or composition is dimethylamine hydrochloride.

In some embodiments, after addition of all buffers and salts, the buffered salt solution is mixed well until all salts are dissolved, and the pH is adjusted using methods known in the art to a pH value of about 8.0 to 9.0. In some embodiments, the pH value is about 8.4-8.8.

In some embodiments, compositions comprise one or more detergents. Exemplary detergents that may be used in the compositions provided herein include nonionic, ionic (anionic, cationic) and zwitterionic detergents. Exemplary such detergents include, but are not limited to, Hecameg (6-0-(N-Heptylcarbamoyl)-methyl-a-D-glucopyranoside), Triton X-200, Brij-58, CHAPS, n-Dodecyl-b-D-maltoside, NP-40, sodium dodecyl sulfate (SDS), TRITON® X-15, TRITON® X-35, TRITON® X-45, TRITON® X-100, TRITON® X-102, TRITON® X-114, TRITON® X-165, TRITON® X-305, TRITON® X-405, TRITON® X-705, Tween® 20 and/or ZWITTERGENT®.

In some embodiments, compositions comprise one or more protein stabilizers. Nonlimiting exemplary protein stabilizers that may be used in the compositions provided herein include BSA, inactive polymerases (such as inactivated Taq polymerase; see, e.g., US Publication No. 2011/0059490), and apotransferrin. Further nonlimiting exemplary stabilizers that may be used in the compositions provided herein include glycerol, trehalose, lactose, maltose, galactose, glucose, sucrose, dimethyl sulfoxide (DMSO), polyethylene glycol, and sorbitol.

In some embodiments, the composition comprises at least one reducing agent. In some embodiments, the reducing agent comprises dithiothreitol (DTT).

In some embodiments, the composition comprises at least one further additive. Further additives can be added, for example, that enhance nucleic acid synthesis from high GC-content templates (e.g. when the GC-content is about 65% or more). The additives may be, for example, ethylene glycol, polyethylene glycol, 1,2-propanediol, ammonium sulfate, dimethyl sulfoxide (DMSO), glycerol, formamide, 7-deaza-GTP, acetamide, or betaine.

In some embodiments, the composition comprises at least one dye. Nonlimiting exemplary dyes that may be used in the compositions provided herein include xylene cyanol FF, tartrazine, phenol red, quinoline yellow, Brilliant Blue, Patent Blue, indigocarmine, amaranth, acid red 1, m-cresol purple, cresol red, neutral red, bromocresol green, acid violet 5, bromo phenol blue, and orange G (see, e.g., U.S. Pat. No. 8,663,925 B2). Additional nonlimiting exemplary dyes are described, e.g., in U.S. Pat. No. 6,942,964. One skilled in the art will appreciate that any dye that does not inhibit nucleic acid synthesis by the polymerases described herein may be used.

In some embodiments, the composition is in a stabilized formulation for long-term storage.

Table 2 provides some non-limiting examples of additional components.

TABLE 2
Additional components that may be comprised in kits or compositions

| Component | Example agent (s) | Range | Unit |
|---|---|---|---|
| Enzyme | DNA polymerases | 0.01-0.5 | u/μl |
| | (including thermophilic | (e.g. 0.04, | |
| | DNA polymerases or | 0.08, 0.1, | |
| | chimeric DNA | 0.16) | |
| | polymerases); RNA | | |
| | polymerases; and/or | | |
| | RNA-dependent DNA | | |
| | polymerases (reverse | | |
| | transcriptases) | | |
| Cofactor for | MgCl₂, MgSO4 | 1-5 | mM |
| polymerase | | (e.g. | |
| | | 1.5, 2) | |
| dNTPs or NTPs | dNTPS: dATP, dCTP, | 0.2-0.3 | mM |
| | dGTP, or dTTP | (e.g. 0.2) | (each) |
| | NTPs: ATP, CTP, | | |
| | GTP, or UTP | | |
| Salt | KCl, KAc | 10-250 | mM |
| (salt may be reduced or | | (e.g. 40, | |
| omitted based on presence | | 50, 70, | |
| of an amine in salt form) | | 100) | |
| Enhancers of nucleic acid | DMSO | 1-10 | % |
| synthesis from high | (NH₄)₂SO₄ | 5-100 | mM |
| GC-content templates | | (e.g. | |
| | | 20, 40) | |
| | 1,2-propanediol | 100-500 | mM |
| | | (e.g. 200, | |
| | | 300, 500) | |
| | Betaine | 0.5-3 | M |
| Buffer | Tris-HCL | 10-100 | mM |
| | | (e.g. | |
| | | 20, 50) | |
| | | 8-9 | pH |
| | | (e.g. | |
| | | 8.4-8.8) | |
| Detergent | Hecameg (6-0-(N- | 0.01-0.5 | % |
| | Heptylcarbamoyl)- | (e.g. 0.15) | |
| | methyl-a-D- | | |
| | glucopyranoside), | | |
| | Triton X-200, Brij-58, | | |
| | CHAPS, n-Dodecyl-b-D- | | |
| | maltoside, NP-40, sodium | | |
| | dodecyl sulfate (SDS), | | |
| | TRITON ® X-15, | | |
| | TRITON ® X-35, | | |
| | TRITON ® X-45, | | |
| | TRITON ® X-100, | | |
| | TRITON ® X-102, | | |
| | TRITON ® X-114, | | |
| | TRITON ® X-165, | | |
| | TRITON ® X-305, | | |
| | TRITON ® X-405, | | |
| | TRITON ® X-705, | | |
| | Tween ® 20 and/or | | |
| | ZWITTERGENTO ® | | |
| Protein stabilizer | Bovine serum | 0.01-2 | mg/ml |
| | albumin (BSA) | (e.g. | |
| | | 0.3, 0.5) | |
| Stabilizers | Sucrose | 1-50 | % |
| | Glycerol | | |
| Preservative | Sodium azide | 0.01-0.02 | % |

3. Hot Start Compositions

In some embodiments, the composition comprising at least one amine and at least one polymerase is a hot start composition. In some such embodiments, the composition comprises a dual hot start composition. In some embodiments, the dual hot start composition comprises at least two different hot start mechanisms that are used to inhibit or substantially inhibit the polymerase activity at a first temperature. Such hot start mechanisms include, but are not limited to, antibodies or combinations of antibodies that block DNA polymerase activity at lower temperatures, antibody mimetics or combinations of antibody mimetics that block DNA polymerase activity at lower temperatures (such as, for example, Affibodies®, see e.g. U.S. Pat. No. 5,831,012), oligonucleotides that block DNA polymerase activity at lower temperatures (such as, for example, aptamers), reversible chemical modifications of the DNA polymerase that dissociate at elevated temperatures, amino acid modifications of the DNA polymerase that provide reduced activity at lower temperatures, fusion proteins that include hyperstable DNA binding domains and topoisomerase, other temperature dependent ligands that inhibit the DNA polymerase, single stranded binding proteins that sequester primers at lower temperatures, or modified primers or modified dNTPs. Hot start compositions, in some embodiments, comprise at least one polymerase with or without a hot start chemical modification, at least one hot start antibody, at least one hot start aptamer, and/or at least one hot start Affibody®.

### III. Methods of Use

In some embodiments, methods for synthesizing a nucleic acid molecule from a sample comprising a template comprise mixing the sample with a composition comprising one or more amines of formula I; providing an enzyme for synthesizing nucleic acid molecules; and incubating said mixture under conditions suitable for synthesis. In some embodiments, synthesizing a nucleic acid molecule is for amplification of the nucleic acid template.

In some embodiments, the template is nucleic acid present in the sample. In some embodiments, the template is DNA or RNA present in the sample.

In some embodiments, the method comprises a purification step for purifying the nucleic acids prior to step of mixing the sample with a composition comprising one or more amines of formula I. In some embodiments, the purification step is performed in the presence of magnetic beads.

A. Improved Yield

In some embodiments, methods comprising use of an amine(s) improve or increase nucleic acid synthesis product yield. In some embodiments, increased yield can be demonstrated by determining the amount of nucleic acid synthesis product obtained in a polymerase (nucleic acid synthesis) reaction comprising amine(s) of formula I, as compared to the amount of product obtained in a reaction carried out under similar reaction conditions, but without amines. The amount of other salts may be reduced based on the presence of an amine in salt form.

The increase in product yield may be at least 10%, 20%, 30%, 40%, 50%, 60%, 70%, 80%, 90%, 100%, 200%, 300%, 400%, or 500% as compared to the amount of product obtained in a reaction carried out under similar reaction conditions, but without amines. The amount of other salts may be reduced based on the presence of an amine in salt form. In some embodiments, the yield may be increased by at least 2, at least 3, at least 4, at least 5 times.

B. Improved Tolerance to Inhibitors

In some embodiments, methods comprising use of an amine(s) improve or increase tolerance to inhibitors. In some embodiments, increased tolerance to inhibitors can be demonstrated by measuring the ability of a polymerase to produce product. In some embodiments, increased tolerance to inhibitors can be demonstrated by determining the amount of product obtained in a polymerase (nucleic acid synthesis) reaction comprising amines of formula I in the presence of certain amount of reaction inhibitor, as compared to the amount of product obtained in a reaction carried out under similar reaction conditions, but without amines. The amount of other salts may be reduced based on the presence of an amine in salt form.

In some embodiments, the increase in product yield in the presence of inhibitors is at least 10%, 20%, 30%, 40%, 50%, 60%, 70%, 80%, 90%, 100%, 200%, 300%, 400%, or 500% as compared to the amount of product obtained in a reaction carried out under similar reaction conditions, but without amines. The amount of other salts may be reduced based on the presence of an amine in salt form. In some embodiments, the yield is increased by at least 2, at least 3, at least 4, at least 5 times.

### IV. Kits

In some embodiments, a kit for use in synthesis of a nucleic acid comprises (i) one or more enzymes for synthesizing nucleic acid molecules or instructions to provide one or more enzymes for synthesizing nucleic acid molecules and (ii) one or more amines of formula I or salts thereof.

In some embodiments, the kit comprises one or more enzymes for synthesizing nucleic acid molecules.

### EXAMPLES

The following examples are provided to illustrate certain disclosed embodiments and are not to be construed as limiting the scope of this disclosure in any way.

### Example 1. Increased PCR Yield and Tolerance to Inhibitors of Taq DNA Polymerase by Amines

The PCR yield and inhibitor tolerance of a Taq DNA (0.04 u/μL) polymerase with an amine (one of diethylamine hydrochloride, diisopropylamine hydrochloride, ethyl(methyl)amine hydrochloride, trimethylamine hydrochloride, dimethylamine hydrochloride) was evaluated by amplifying a 727 bp PCR fragment in the presence of various amounts of xylan, urea, and sodium citrate.

A 727 bp fragment was amplified from 62.5 ng of human genomic DNA template in 50 μl PCR reactions in the following PCR buffers:
PCR buffer 1: 10 mM TRIS-HCl, pH 8.8; 0.08% (v/v) Nonidet P40; 50 mM KCl; 2 mM MgCl₂, and 0.2 mM dNTPs.
PCR buffer 2: 10 mM TRIS-HCl, pH 8.8; 0.08% (v/v) Nonidet P40; 40 mM KCl; 10 mM amine (one of diethylamine hydrochloride, diisopropylamine hydrochloride, ethyl(methyl)amine hydrochloride, trimethylamine hydrochloride, dimethylamine hydrochloride); 2 mM MgCl₂, and 0.2 mM dNTPs.
PCR buffer 3: 10 mM TRIS-HCl, pH 8.8; 0.08% (v/v) Nonidet P40; 10 mM KCl; 40 mM amine (one of diethylamine hydrochloride, diisopropylamine hydrochloride, ethyl(methyl)amine hydrochloride, trimethylamine hydrochloride, dimethylamine hydrochloride); 2 mM MgCl₂, and 0.2 mM dNTPs.
PCR buffer 4: 10 mM TRIS-HCl, pH 8.8; 0.08% (v/v) Nonidet P40; 50 mM amine (one of diethylamine hydrochloride, diisopropylamine hydrochloride, ethyl(methyl)amine hydrochloride, trimethylamine hydrochloride, dimethylamine hydrochloride); 2 mM MgCl₂, and 0.2 mM dNTPs.

A forward primer (SEQ ID No: 1) and a reverse primer (SEQ ID No: 2) were used with the PCR program described in Table 3. PCR products were detected by agarose gel electrophoresis followed by ethidium bromide staining

TABLE 3
PCR program for determining PCR yield and
tolerance to inhibitors with Taq DNA polymerase

| Temperature | Time | |
|---|---|---|
| 95° C. | 3 min | |
| 95° C. | 30 sec | Cycles |
| 58° C. | 30 sec | repeated |
| 72° C. | 45 sec | 30 times |

A. Xylan

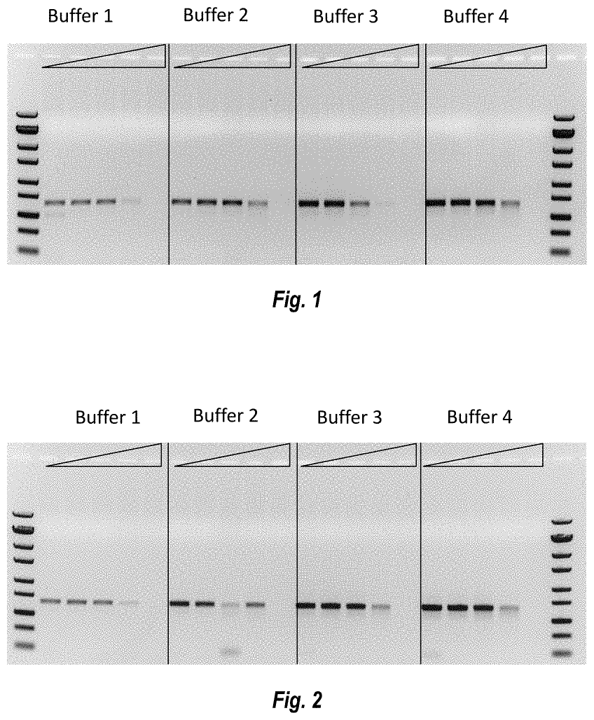

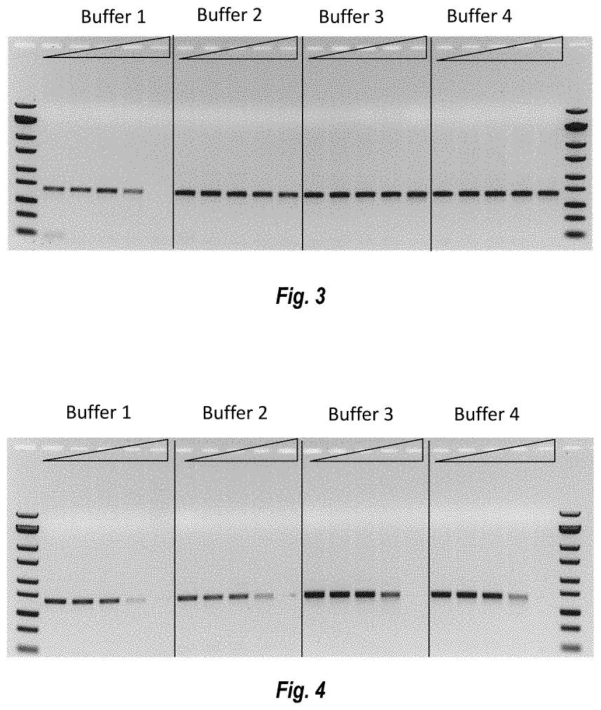

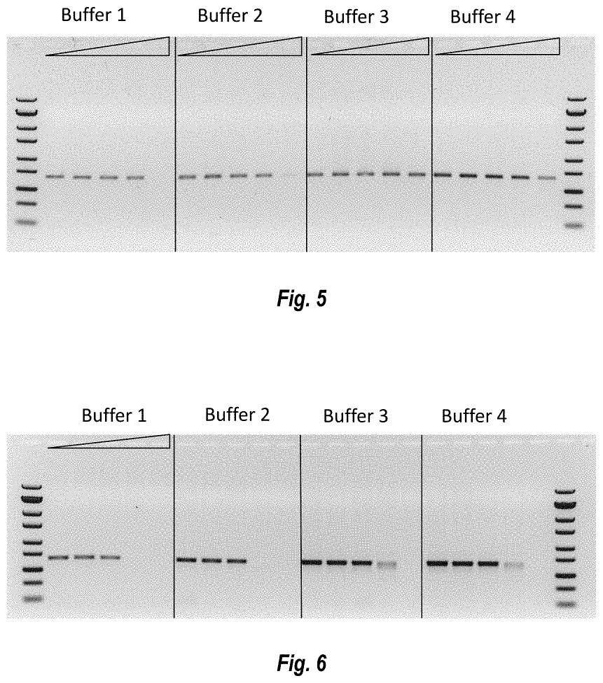

A 727 bp fragment was amplified by the Taq DNA polymerase in the presence of 0 ng/μl, 19 ng/μl, 39 ng/μl, 77 ng/μl, or 154 ng/μl of xylan. PCR product yield was higher in buffers containing diethylamine hydrochloride (FIG. 1), diisopropylamine hydrochloride (FIG. 2), trimethylamine hydrochloride (FIG. 4) when compared to PCR buffer 1 without amines PCR buffers containing ethyl(methyl)amine hydrochloride (FIG. 3) and dimethylamine hydrochloride (FIG. 5) in addition to the increased PCR product yield, also showed two-times greater tolerance to xylan (154 ng/μl) compared with PCR buffer 1 (77 ng/μl).

B. Urea

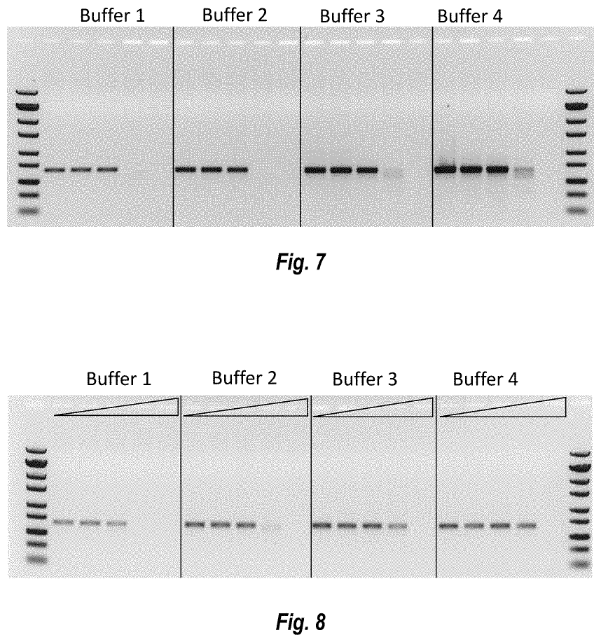

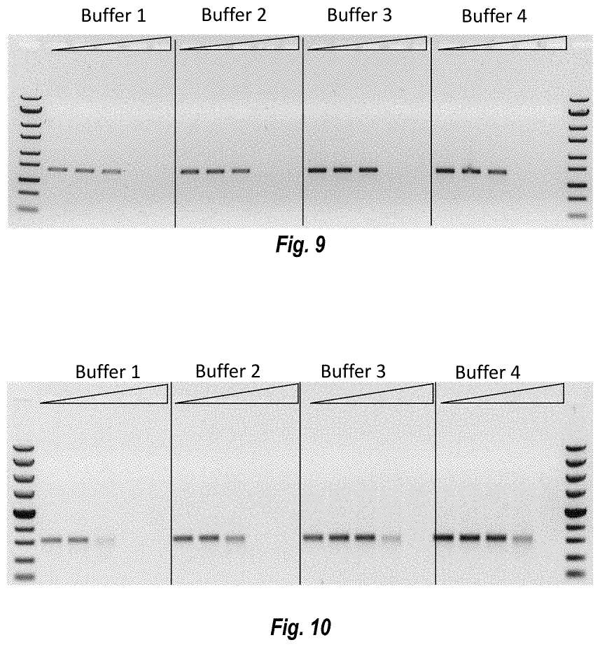

A 727 bp fragment was amplified by the Taq DNA polymerase in the presence of 0 mM, 15 mM, 37 mM, 92 mM, or 230 mM of urea. Detectable PCR product was observed at up to 92 mM of urea for buffers containing diethylamine hydrochloride (FIG. 6), diisopropylamine hydrochloride (FIG. 7), ethyl(methyl)amine hydrochloride (FIG. 8), dimethylamine hydrochloride (FIG. 10) indicating 2.5 times greater tolerance to urea compared with PCR buffer 1 (37 mM). The reaction with PCR buffers containing trimethylamine hydrochloride showed the same resistance to urea as PCR buffer 1 (FIG. 9). These results also showed increased PCR product yield of a Taq DNA polymerase by amines (such as diethylamine hydrochloride, diisopropylamine hydrochloride, ethyl(methyl)amine hydrochloride, trimethylamine hydrochloride, dimethylamine hydrochloride) compared with PCR buffer 1 (FIG. 6-10).

C. Sodium Citrate

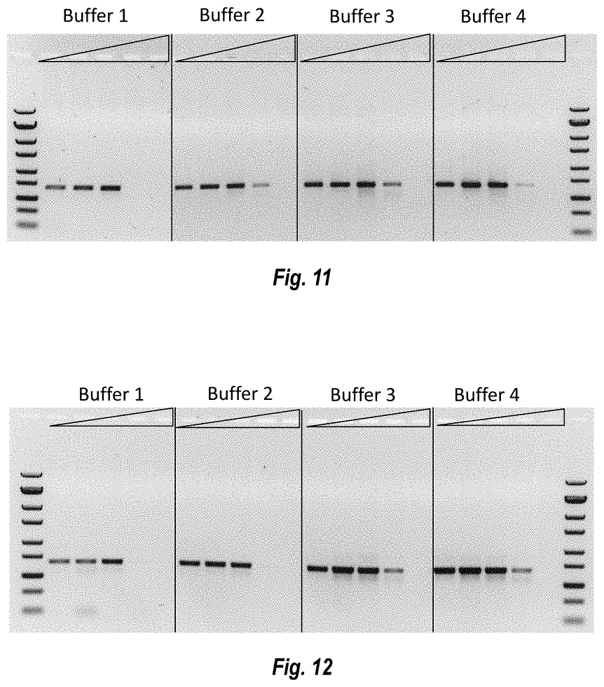

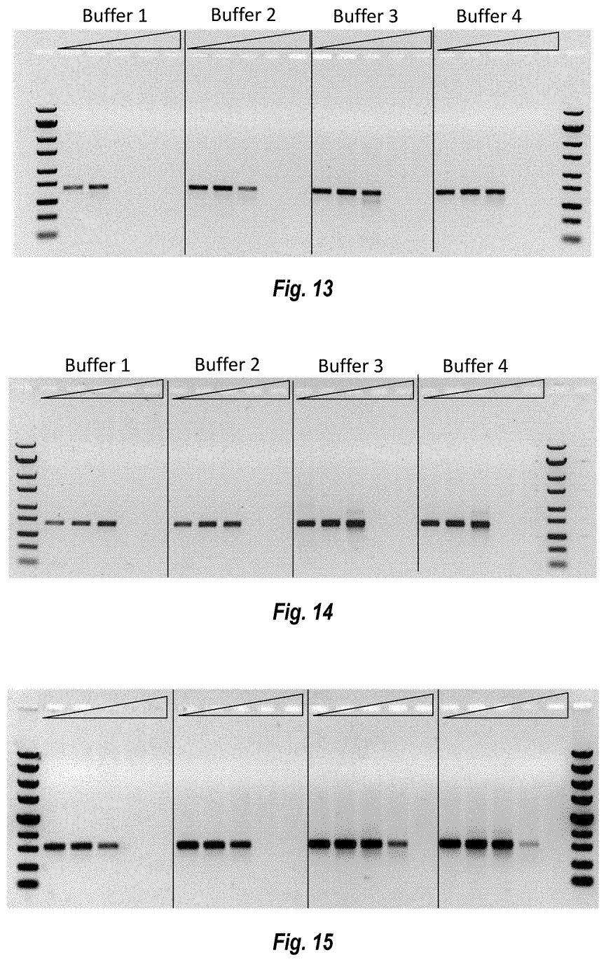

A 727 bp fragment was the Taq DNA polymerase in the presence of 0%, 0.02%, 0.04%, 0.05%, or 0.08% of sodium citrate. Higher PCR product yield was observed in PCR buffers containing an amine (such as diethylamine hydrochloride, diisopropylamine hydrochloride, ethyl(methyl)amine hydrochloride, trimethylamine hydrochloride, dimethylamine hydrochloride) when compared with PCR buffer 1. Reactions in PCR buffers containing ethyl(methyl)amine hydrochloride (FIG. 13) or trimethylamine hydrochloride (FIG. 14) showed the same tolerance to sodium citrate as PCR buffer 1-0.04%, while PCR buffers with diethylamine hydrochloride (FIG. 11), diisopropylamine hydrochloride (FIG. 12), dimethylamine hydrochloride (FIG. 15) showed 1.25 times greater tolerance to sodium citrate (0.05%) compared to PCR buffer 1.

Thus, an amine (such as diethylamine hydrochloride, diisopropylamine hydrochloride, ethyl(methyl)amine hydrochloride, trimethylamine hydrochloride, dimethylamine hydrochloride) increased yield and/or increased tolerance of Taq DNA polymerase in the presence of xylan, urea, and sodium citrate.

Experiments with mutant Taq DNA polymerase where the reaction buffer contained 70 mM, 80 mM or 90 mM of KCl showed increased PCR product yield when part or all of the potassium salt in the reaction buffer was replaced with dimethylamine hydrochloride (data not shown).

### Example 2. Increased PCR Yield and Tolerance for Inhibitors of Platinum SuperFi DNA Polymerase by Amines

Yield and tolerance for urea in PCR master mix with different amines compared to the control master mix (with KCl) was evaluated by amplification of a 1 kb fragment from 42 ng of human genomic DNA template in 50 μl PCR reactions.

PCR master mix (1×) composition was 0.16 u/μL Platinum SuperFi DNA polymerase in 1× SuperFi buffer. In some experiments, 110 mM of KCl was substituted for solution comprising amines. In addition, the master mix contained either water or 0.14 M, 0.28 M, 0.42 M, 0.56 M, 0.70 M, 0.84 M, or 0.98 M urea to test the effect of urea contamination. The following amines were tested: diethylamine hydrochloride, ethyl(methyl)amine hydrochloride, and dimethylamine hydrochloride. The forward primer used was SEQ ID No: 3 and the reverse primer was SEQ ID No: 4.

The PCR program used for the experiment is described in Table 4.

TABLE 4
PCR program for determining PCR yield and
tolerance to inhibitors with SuperFi polymerase

| Temperature | Time | |
|---|---|---|
| 98° C. | 30 sec | |
| 98° C. | 5 sec | Cycles |
| 63° C. | 15 sec | repeated |
| 72° C. | 30 sec | 25 times |
| 72° C. | 5 min | |

Products were detected by electrophoresis in a 1% TAE gels followed by staining with ethidium bromide.

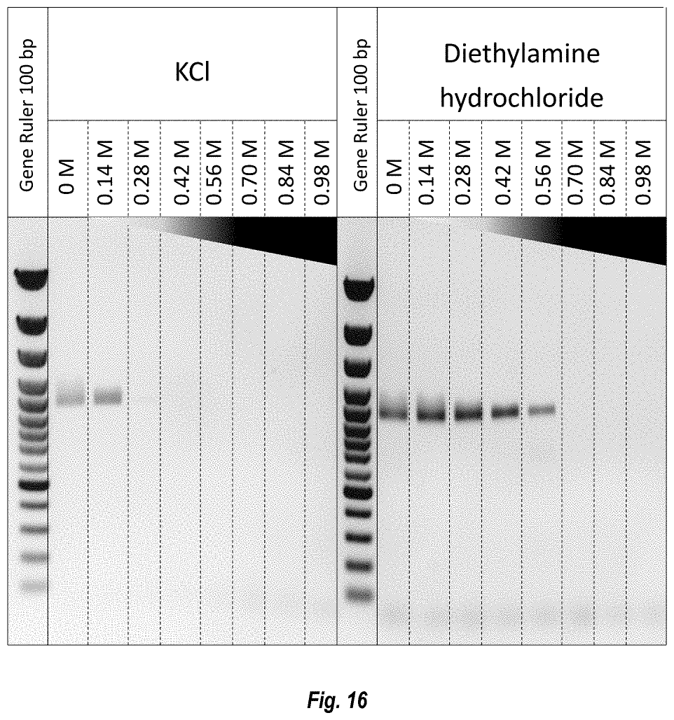

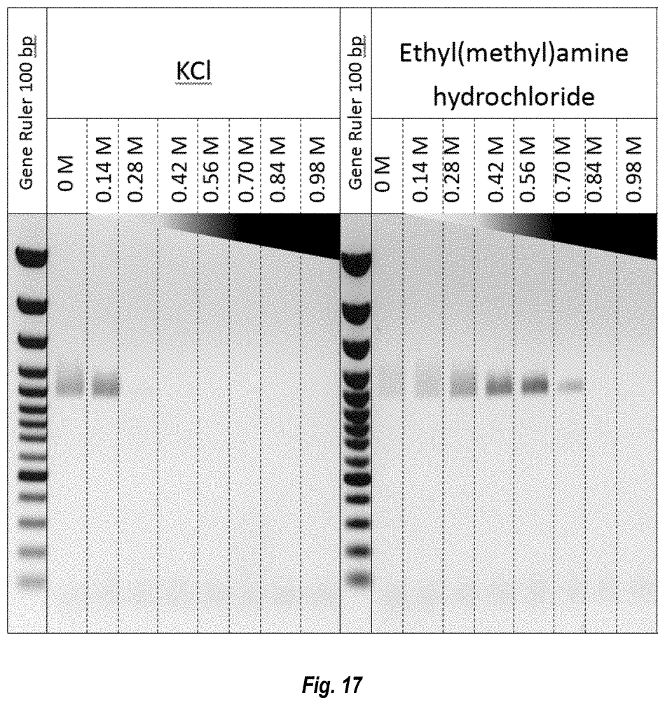

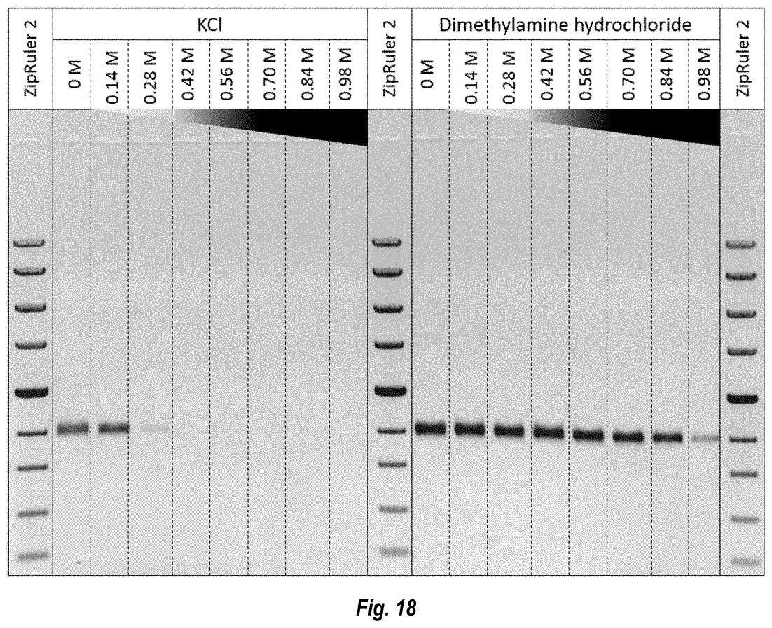

In PCR reactions containing dimethylamine hydrochloride sufficient amounts of product were observed even in presence of 0.98 mol/L urea (FIG. 18), while only 0.14 mol/L of urea was tolerated by the control master mix. Increased tolerance to urea when compared to PCR buffer with KCl was also observed when using diethylamine hydrochloride (FIG. 16) and ethylamine hydrochloride (FIG. 17) as the amine. Moreover, PCR yield was much higher in presence of dimethylamine hydrochloride (FIG. 18) or diethylamine hydrochloride (FIG. 16).

These data indicate that amines improve tolerance to urea and increase PCR yield in the presence of urea.

Experiments with SuperFi DNA polymerase where the reaction buffer contained 70 mM, 80 mM or 90 mM of KCl showed increased PCR product yield when part or all of the potassium salt in the reaction buffer was replaced with dimethylamine hydrochloride (data not shown).

### Example 3. Increased PCR Yield and Tolerance for Magnetic Beads of Platinum SuperFi DNA Polymerase by Amines

PCR yield and tolerance for magnetic beads in PCR master mix with different amines compared to the control master mix (with KCl) was evaluated by amplification of a 1 kb fragment from 42 ng of human genomic DNA template. PCR was performed in 50 μl PCR reactions in the presence of increasing amounts of magnetic beads.

The following amines were tested: diethylamine hydrochloride, ethylamine hydrochloride, trimethylamine hydrochloride and dimethylamine hydrochloride. The PCR master mix (1×) concentration of KCl (110 mM) was substituted for solution comprising an amine.

Agencourt XP magnetic beads were used in the experiment. From 5 to 35 μl of magnetic beads were washed 2 times with 80% EtOH and dried (an imitation of a protocol for purifying nucleic acids). Dried magnetic beads were resuspended in 50 μl of PCR reaction (complete with PCR master mix, DNA matrix, and primers). The forward primer was SEQ ID No: 3, and the reverse primer was SEQ ID No: 4. Table 4 provides the PCR program for the experiment.

Products were detected by electrophoresis in a 1% TAE gels followed by staining with ethidium bromide.

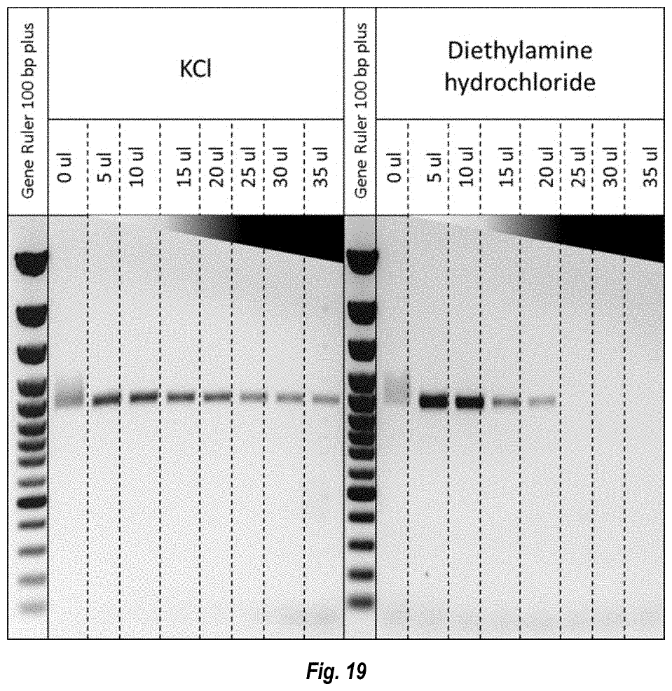

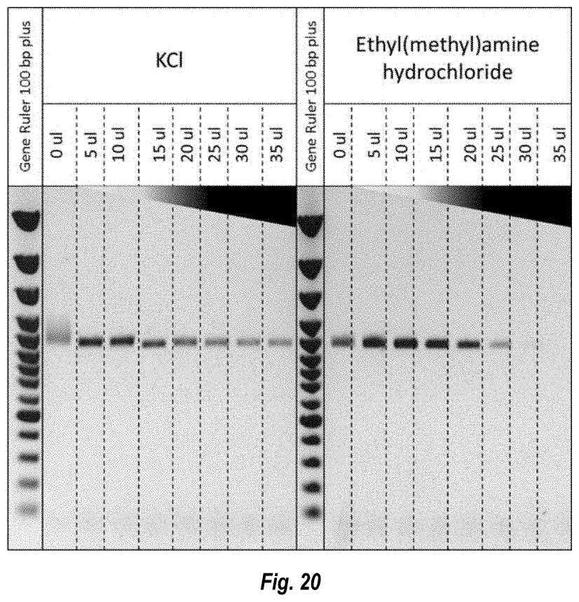

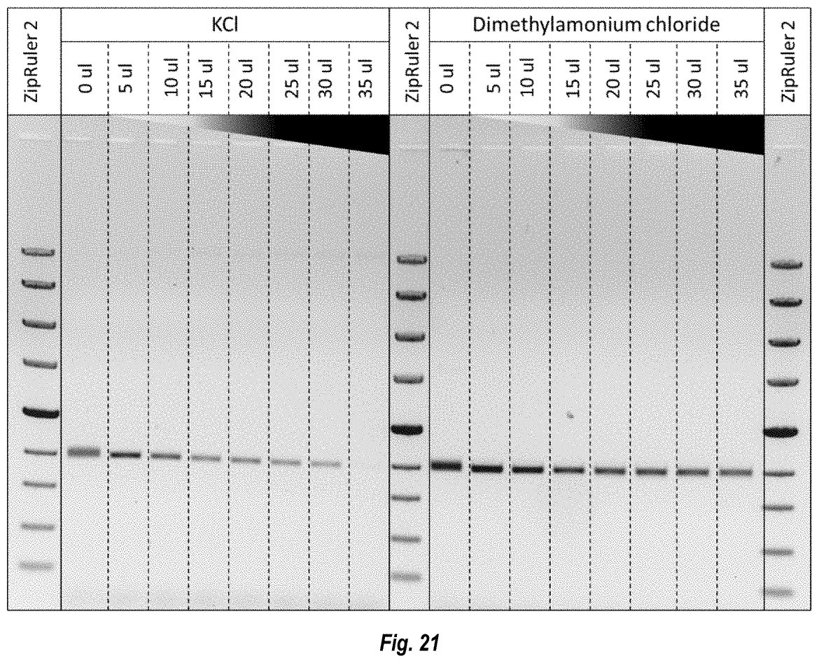

Increased yield of PCR product when using up to 20 μl of Agencourt XP magnetic beads in PCR reaction was observed when the PCR solution comprised diethylamine hydrochloride (FIG. 19), ethyl(methyl)amine hydrochloride (FIG. 20), trimethylamine hydrochloride (data not shown), or dimethylamine hydrochloride (FIG. 21). Dimethylamine hydrochloride also increased the tolerated amount of magnetic beads in the PCR reaction (FIG. 21).

Experiments with SuperFi DNA polymerase where the reaction buffer contained 70 mM, 80 mM or 90 mM of KCl showed increased PCR product yield in the presence of magnetic beads when part or all of the potassium salt in the reaction buffer was replaced with dimethylamine hydrochloride (data not shown).

Thus, amines (such as diethylamine hydrochloride, ethyl(methyl)amine hydrochloride, trimethylamine hydrochloride, or dimethylamine hydrochloride) increased PCR yield with Platinum SuperFi polymerase in the presence of magnetic beads.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method for improving nucleic acid synthesis product yield during nucleic acid synthesis from a nucleic acid template comprising:
a. mixing a sample comprising the nucleic acid template with a composition comprising one or more amines of formula I: 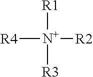
or salts thereof wherein
R1 is H;
R2 is chosen from alkyl, alkenyl, alkynyl, or (CH2)n-R5, wherein n=1 to 3, and
R5 is aryl, amino, thiol, mercaptan, phosphate, hydroxy, or alkoxy; and
R3 and R4 may be the same or different and are independently chosen from H or alkyl, with the proviso that if R2 is (CH2)n-R5, then at least one of R3 and/or R4 is alkyl;
b. providing one or more enzymes for synthesizing nucleic acid molecules; and
c. incubating said mixture under conditions suitable for synthesis.

2. The method of claim 1, wherein the one or more amines of formula I and the one or more enzymes for synthesizing nucleic acid molecules are provided simultaneously.

3. The method of claim 1 or 2, wherein the synthesis is for amplification of the nucleic acid template.

4. The method of any one of claims 1 to 3, wherein the one or more amines of formula I and/or the one or more enzymes for synthesizing nucleic acid molecules is in a stabilized formulation for long-term storage.

5. The method of any one of claims 1 to 4, wherein the one or more amines of formula I and/or the one or more enzymes for synthesizing nucleic acid molecules are provided in a formulation that comprises a stabilizer and/or detergent.

6. The method of any one of claims 1 to 5, wherein the sample comprises one or more nucleic acid synthesis inhibitors selected from a polyanion, a chaotropic agent, a protein, an organic compound, a chelator, an organic solvent, a metal ion, or a nucleic acid intercalating dye.

7. The method of claim 6, wherein said polyanion is heparin or xylan, and/or said chaotropic agent is sodium dodecyl sulfate or urea, and/or said protein is collagen, heme or heme-containing protein, and/or said organic compound is humic acid or bile salts, and/or said chelator is citrate or EDTA, and/or said solvent is ethanol or isopropanol, and/or the metal ion is calcium.

8. The method of any one of claims 1 to 5, wherein the method is performed in the presence of magnetic beads.

9. The method of claim 8, wherein the magnetic beads are carboxylated magnetic beads.

10. The method of claim 9, wherein the carboxylated magnetic beads are Agencourt® AMPure® XP (Beckman Coulter, Inc), Sera-Mag™ SpeedBeads™ (GE Healthcare Life Sciences), MyOne carboxylated beads (DynaBeads), or Mag-Bind® RXNPure (Omega Bio-tek, Inc) beads.

11. The method of any one of claims 1 to 10, wherein the method comprises a purification step for purifying the nucleic acids prior to step a.

12. The method of claim 11, wherein said purification step is performed in the presence of magnetic beads.

13. The method of any one of claims 1 to 12, wherein the composition further comprises one or more additional components chosen from (i) one or more nucleic acid molecules; (ii) one or more nucleotides; (iii) one or more buffering salts; and (iv) one or more cofactors.

14. The method of claim 13, wherein the one or more nucleic acid molecules comprise RNA or DNA.

15. The method of claim 13 or 14, wherein the one or more nucleic acid molecules comprise at least one primer.

16. The method of any one of claims 13 to 15, wherein the one or more nucleotides comprise dNTPs or NTPs.

17. The method of any one of claims 13 to 16, wherein the one or more buffering salts comprise acetate, sulfate, hydrochloride, or phosphate or free acid forms of Tris-(hydroxymethyl)aminomethane (TRIS®).

18. The method of any one of claims 13 to 17, wherein the one or more cofactor comprise a magnesium salt.

19. The method of any one of claims 1 to 18, wherein the composition further comprises one or more additional additives.

20. The method of claim 19, wherein the additional additive comprises a salt.

21. The method of claim 20, wherein the salt comprises a potassium salt.

22. The method of claim 21, wherein the potassium salt comprises KCl.

23. The method of claim 22, wherein the KCl concentration of the composition may be reduced or KCl may be omitted based on the presence of an amine.

24. The method of any one of claims 19 to 23, wherein the additional additive comprises a detergent.

25. The method of claim 24, wherein the detergent comprises Hecameg (6-0-(N-Heptylcarbamoyl)-methyl-a-D-glucopyranoside), Triton X-200, Brij-58, CHAPS, n-Dodecyl-b-D-maltoside, NP-40, sodium dodecyl sulfate (SDS), TRITON® X-15, TRITON® X-35, TRITON® X-45, TRITON® X-100, TRITON® X-102, TRITON® X-114, TRITON® X-165, TRITON® X-305, TRITON® X-405, TRITON® X-705, Tween® 20 and/or ZWITTERGENT®.

26. The method of any one of claims 19 to 25, wherein the additional additive comprises at least one protein stabilizer.

27. The method of claim 26, wherein the protein stabilizer comprises bovine serum albumin (BSA), an inactive polymerase, or apotransferrin.

28. The method of any one of claims 19 to 27, wherein the additional additive comprises at least one reducing agent.

29. The method of claim 28, wherein the reducing agent comprises dithiothreitol (DTT).

30. The method of any one of claims 19 to 29, wherein the additional additive comprises an agent that enhances nucleic acid synthesis from high GC-content templates.

31. The method of claim 30, wherein the agent that enhances nucleic acid synthesis from high GC-content templates comprises ethylene glycol, polyethylene glycol, 1,2-propanediol, ammonium sulfate, dimethyl sulfoxide (DMSO), glycerol, formamide, 7-deaza-GTP, acetamide, or betaine.

32. The method of any one of claims 19 to 31, wherein the additional additive comprises a dye.

33. The method of claim 32, wherein the dye comprises xylene cyanol FF, tartrazine, phenol red, quinoline yellow, Brilliant Blue, Patent Blue, indigocarmine, acid red 1, m-cresol purple, amaranth, cresol red, neutral red, bromocresol green, acid violet 5, bromo phenol blue, or orange G.

34. The method of any one of claims 19 to 33, wherein the additional additive comprises glycerol, trehalose, lactose, maltose, galactose, glucose, sucrose, dimethyl sulfoxide (DMSO), polyethylene glycol, or sorbitol.

35. The method of any one of claims 1 to 34, wherein the composition comprises a hot start composition.

36. The method of any one of claims 1 to 35, wherein the one or more enzyme for synthesizing nucleic acid is chosen from a DNA polymerase, an RNA polymerase, or a reverse transcriptase.

37. The method of claim 36, wherein the DNA polymerase comprises Phi29, Bsm, Bst, T4, T7, DNA Pol I, or Klenow Fragment; or mutants, variants and derivatives thereof.

38. The method of claim 36, wherein the DNA polymerase comprises a thermophilic DNA polymerase.

39. The method of claim 38, wherein the thermophilic DNA polymerase comprises Taq, Tbr, Tfl, Tth, Tli, Tfi, Tne, Tma, Pfu, Pwo, Kod, VENT™, DEEPVENT™ DNA polymerase; Phusion DNA polymerase; Phusion U DNA polymerase; SuperFi DNA polymerase; SuperFi U DNA Polymerase; or mutants, variants and derivatives thereof.

40. The method of any one of claims 36 to 39, wherein the DNA polymerase comprises a chimeric DNA polymerase.

41. The method of claim 40, wherein the chimeric DNA polymerase comprises a sequence nonspecific double stranded DNA (dsDNA) binding domain.

42. The method of claim 41, wherein the dsDNA binding domain comprises Sso7d from Sulfolobus solfataricus; Sac7d, Sac7a, Sac7b, and Sac7e from S. acidocaldarius; and Ssh7a and Ssh7b from Sulfolobus shibatae; Pae3192; Pae0384; Ape3192; HMf family archaeal histone domains; or an archaeal proliferating-cell nuclear antigen (PCNA) homolog.

43. The method of claim 36, wherein the RNA polymerase comprises SP6, T7, or T3 RNA polymerase, or mutants, variants, or derivatives thereof.

44. The method of claim 36, wherein the reverse transcriptase comprises M-MLV reverse transcriptase, RSV reverse transcriptase, AMV reverse transcriptase, RAV reverse transcriptase, MAV reverse transcriptase, HIV reverse transcriptase, or mutants, variants, and derivatives thereof.

45. The method of any one of claims 1 to 42, wherein the method is for polymerase chain reaction (PCR).

46. The method of any one of claims 1 to 45, wherein R2 is an alkyl.

47. The method of claim 46, wherein the alkyl is a C1-05 (branched or linear) alkyl.

48. The method of claim 46 or 47, wherein the alkyl is a C1-C3 alkyl.

49. The method of any one of claims 1 to 48, wherein R3 and/or R4 is an H.

50. The method of any one of claims 1 to 49, wherein R3 and/or R4 is an alkyl.

51. The method of claim 50, wherein the alkyl is a C1-C5 (branched or linear) alkyl.

52. The method of claim 50 or 51, wherein the alkyl is a C1-C3 alkyl.

53. The method of any one of claims 1 to 52, wherein the composition comprises a salt form of the one or more amines of formula I.

54. The method of claim 53, wherein the salt form comprises a chloride, sulfate, or acetate salt.

55. The method of any one of claims 1 to 54, wherein said composition comprises one amine of formula I or salts thereof.

56. The method of any one of claims 1 to 54, wherein said composition comprises two or more amines of formula I or salts thereof.

57. The method of any one of claims 1 to 54, wherein said composition comprises three or more amines of formula I or salts thereof.

58. The method of any one of claims 1 to 54, wherein said composition comprises four or more amines of formula I or salts thereof.

59. The method of any one of claims 1 to 58, wherein the concentration of the one or more amines is 10-250 mM.

60. The method of claim 59, wherein the concentration of the one or more amines is 50-110 mM.

61. The method of any one of claims 1 to 60, wherein at least one amine of formula I is selected from dimethylamine hydrochloride, diethylamine hydrochloride, diisopropylamine hydrochloride, ethyl(methyl)amine hydrochloride, or trimethylamine hydrochloride.

62. The method of any one of claims 1 to 61, wherein the nucleic acid synthesis yield is increased by at least 10%, 20%, 30%, 40%, 50%, 60%, 70%, 80%, 90%, 100%, 200%, 300%, 400%, or 500%.

63. The method of any one of claims 6 to 61, wherein the nucleic acid synthesis yield in the presence of nucleic acid synthesis inhibitors is increased by at least 10%, 20%, 30%, 40%, 50%, 60%, 70%, 80%, 90%, 100%, 200%, 300%, 400%, or 500%.

64. A kit for use in synthesis of a nucleic acid molecule, said kit comprising, (i) one or more enzymes for synthesizing nucleic acid molecules or instructions to provide one or more enzymes for synthesizing nucleic acid molecules and (ii) one or more amines of formula I: 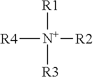
or salts thereof wherein
R1 is H;
R2 is chosen from alkyl, alkenyl, alkynyl, or (CH2)n-R5, wherein n=1 to 3, and R5 is aryl, amino, thiol, mercaptan, phosphate, hydroxy, or alkoxy; and
R3 and R4 may be the same or different and are independently chosen from H or alkyl, with the proviso that if R2 is (CH2)n-R5, then at least one of R3 and/or R4 is alkyl.

65. A composition for improving nucleic acid synthesis product yield comprising one or more enzymes for synthesizing nucleic acid molecules and one or more amines of formula I: 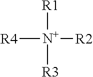
or salts thereof wherein
R1 is H;
R2 is chosen from alkyl, alkenyl, alkynyl, or (CH2)n-R5, wherein n=1 to 3, and
R5 is aryl, amino, thiol, mercaptan, phosphate, hydroxy, or alkoxy; and
R3 and R4 may be the same or different and are independently chosen from H or alkyl, with the proviso that if R2 is (CH2)n-R5, then at least one of R3 and/or R4 is alkyl.


SEQUENCE LISTING (4 sequences)

SEQ ID NO: 1 - DNA, 19 bp, Artificial Sequence
  source: /note=“Description of Artificial Sequence Synthetic primer”
gccctgttcacccgttctt

SEQ ID NO: 2 - DNA, 18 bp, Artificial Sequence
  source: /note=“Description of Artificial Sequence Synthetic primer”
gtggcaacatggcccttc

SEQ ID NO: 3 - DNA, 20 bp, Artificial Sequence
  source: /note=“Description of Artificial Sequence Synthetic primer”
cagggcagggagttgaagtt

SEQ ID NO: 4 - DNA, 21 bp, Artificial Sequence
  source: /note=“Description of Artificial Sequence Synthetic primer”
caggttggtattgccttctgg